(19) United States
(12) Patent Application Publication
(10) Pub. No.: US 2023/0077300 A1
(43) Pub. Date: Mar. 9, 2023

(54) METHODS FOR TREATING OR AMELIORATING AUTISM AND RELATED DISORDERS

(71) Applicant: Scioto BioSciences, Inc., Indianapolis, IN (US)

(72) Inventor: Mark Heiman, Indianapolis, IN (US)

(21) Appl. No.: 17/792,329

(22) PCT Filed: Jan. 13, 2021
(86) PCT No.: PCT/US21/13280

§ 371 Date: Jul. 12, 2022

Related U.S. Application Data
(60) Provisional application No. 62/960,331, filed on Jan. 13, 2020.
(60) Provisional application No. 62/960,328, filed on Jan. 13, 2020.

(51) Int. Cl. A61K 35/747; A61K 47/26; A61P 1/00; A61P 25/24

(52) U.S. Cl. CPC A61K 35/747; A61K 47/26; A61P 1/00; A61P 25/24

(57) ABSTRACT

The present disclosure relates in part to methods for treating or preventing a disease using a bacterial formulation that comprises a probiotic bacterium, a biocompatible microsphere, and/or a prebiotic.

### RELATED APPLICATIONS

This application claims priority to U.S. Ser. No. 62/960,328 filed Jan. 13, 2020, and U.S. Ser. No. 62/960,331 filed Jan. 13, 2020, the contents of which are incorporated herein by reference.

### BACKGROUND

Probiotics are live microbes that when ingested in high enough quantities confer a health benefit for the host (Food and Agriculture Organization of the United Nations and World Health Organization, “Health and Nutritional Properties of Probiotics in Food Including Powdered Milk with Live Bacteria” (2001)). As a result, probiotics are gaining traction as a viable option for treating disease (Hemarajata, P. et al. (2013); Therap Adv Gastroenterol.; 6(1):39-51). When bacteria are already in the activated form of a biofilm (a surface and/or self-adhered community) as opposed to planktonic (free-living), they more readily establish and persist. The positive effects of probiotic bacteria can be enhanced by providing them in an activated state; this can readily be accomplished by exposing the bacteria to the surface of a biocompatible and non-toxic microsphere.

Oxytocin is a hypothalamus derived, posterior pituitary stored nonapeptide which has gained recent interest as an important neuropsychiatric and metabolic hormone beyond its classic role in lactation and parturition. Oxytocin is central to the recognition of complex social cognition and behaviors, such as attachment, social exploration, and recognition. In healthy humans, oxytocin binds to receptors in social brain regions such as amygdala and anterior cingulate cortex (Boccia, M. et al. (2013); Neuroscience.; 253, 155-164). It is a key component of the network regulating social brain functions such as modulation of social stress, emotion recognition and memory formation (Meyer-Lindenberg, A. et al. (2011); Nat. Rev. Neruosci.; 12, 524). In autistic disorder, intranasal administration of oxytocin in some studies reported that it improves emotion recognition. Further, oxytocin does not cross the blood-brain barrier inviting delivery by intranasal administration. However, intranasal oxytocin is unable to achieve sustained physiological levels of pulsatile oxytocin signaling. Autistic patients may also have an increased incidence of gastrointestinal disorders.

Additionally, in many patients, use of antibiotics is accompanied by a side effect of gastrointestinal disorders that range from the mild (such as upset stomach, nausea, diarrhea, and vomiting) to the severe (such as abdominal pain, pseudomonas colitis). These side effects can be even more significant in pediatric and elderly (patients who over 65 years old) population undergoing antibiotic treatment. These antibiotic regimen associated-gastrointestinal side effects have been reported to arise from the elimination of the beneficial flora which is necessary to facilitate normal digestive processes of the gut. Scientific studies have shown that use of antibiotics results in disruption of the normal ecology of the gut, causing a decrease in the population of beneficial bacteria and an abnormal and harmful increase in harmful bacteria.

There is therefore a need for safe, effective therapies to treat or ameliorate autism, as well as gastrointestinal disorders related to autism and/or antibiotic use.

### SUMMARY

This disclosure is directed in part to a method of treating or ameliorating an autism spectrum disorder in a subject in need thereof, comprising administering to the subject a therapeutically effective amount of a composition comprising Lactobacillus reuteri, a biocompatible microsphere, and optionally a prebiotic (e.g., a composition comprising a dextranomer microparticle; L. reuteri with deposit # ATCC 23272; and a prebiotic) Contemplated autism spectrum disorders for treatment with the provided methods may include autistic disorder, Asperger syndrome, Heller's syndrome, Rett syndrome, and pervasive developmental disorder, and not otherwise specified (PDD-NOS).

Provided herein is a method of treating a developmental disorder and/or autism, for example, which may be, for example, associated with preterm birth in a preterm infant, comprising administering to a patient in need thereof, e.g., preterm infant, a therapeutically effective amount of a composition comprising Lactobacillus reuteri and a pharmaceutically acceptable carrier. In certain embodiments, the composition comprises a dextranomer microparticle, L. reuteri, ATCC 23272, and a prebiotic. In certain embodiments, upon administration of at least one daily dose of the composition to the subject, the subject has an upregulation in oxytocin levels.

In an embodiment, provided herein is method of treating gastrointestinal inflammation in a patient due to administration of an antibiotic regimen, comprising administering to the patient a therapeutically effective amount of a composition comprising Lactobacillus reuteri, a biocompatible microsphere, and/or a prebiotic. For example, provided herein is method of treating a patient having or expected to have gastrointestinal inflammation associated with administration of an oral antibiotic regimen, comprising administering to the patient a therapeutically effective amount of a composition comprising Lactobacillus reuteri, a biocompatible microsphere, and/or a prebiotic. Such a composition may be administered before administration of the antibiotic regimen, during the administration of the antibiotic regimen, and/or after administration of the antibiotic regimen. A method of substantially preventing or decreasing an oral antibiotic-associated adverse effect in a subject in need thereof, is also provided in an embodiment, comprising administering a therapeutically effective amount of a composition comprising Lactobacillus reuteri, a biocompatible microsphere, wherein the subject is undergoing treatment with the oral antibiotic.

Also contemplated and provided herein is a method of treating depression or an anxiety disorder in a subject in need thereof, the method comprising administering to the subject a therapeutically effective amount of a composition comprising Lactobacillus reuteri, a biocompatible microsphere, and/or a prebiotic. For example, provided herein is a method of treating one or more of clinical depression, postnatal or postpartum depression, obsessive-compulsive disorder, post-traumatic stress disorder, bipolar disorder, atypical depression, melancholic depression, Psychotic Major Depression (PMD), catatonic depression, Seasonal Affective Disorder (SAD), dysthymia, double depression, Depressive Personality Disorder (DPD), Recurrent Brief Depression (RBD), minor depressive disorder, bipolar disorder or manic depressive disorder, treatment-resistant depression, refractory depression, suicidality, suicidal ideation, and suicidal behavior using the disclosed methods. In certain embodiments, the patient has postnatal or postpartum depression.

In some embodiments, contemplated methods of treatment herein may be directed to a male or a female subject in need. In some embodiments, contemplated methods of treatment herein may be directed to an infant, a child, or an adult. In certain embodiments, contemplated methods of treatment herein comprises administering a therapeutically effective amount of a composition comprising Lactobacillus reuteri and a pharmaceutically acceptable carrier, wherein the composition is administered as a unit dose. In certain other embodiments, the composition is administered in multiple doses. In certain embodiments, the composition is administered at least daily. In certain embodiments, the composition is administered once.

### BRIEF DESCRIPTION OF FIGURES

FIGS. 1A-B show the stomach content dry mass in saline-treated female and male rat pups, respectively. FIG. 1C shows total stomach calories in day 17 (D17) and day 21 (D21) male pups. FIGS. 1D-E show the caloric density in saline-treated female and male pups, respectively.

FIGS. 2A-B compare the stomach content dry mass in Formulation A-treated and [symbol] saline-treated pups (data from D21 female and D21 male pups are shown in FIGS. 2A and 2B, respectively). FIGS. 2C-D compare the stomach caloric content in Formulation A-treated and [symbol] saline-treated pups (data from D21 female and D21 male pups are shown in FIGS. 2C and 2D, respectively). FIGS. 2E-F compare the stomach lactose content in Formulation A-treated and [symbol] saline-treated pups (data from D21 female and D21 male pups are shown in FIGS. 2E and 2F, respectively).

FIG. 3A shows the stomach content pH in saline-treated female rat pups. FIGS. 3B-C compare the stomach content pH in Formulation A-treated and [symbol] saline-treated pups (data from D20 female and D21 male pups are shown in FIGS. 3B and 3C, respectively).

FIG. 4A shows blood glucose and jejunum glucose levels. FIG. 4B shows jejunum protein concentration in female rat pups.

FIGS. 5A-C compare the serum peptidoglycan levels in 16 day old female rat pups (24 hours) (FIG. 5A), (48 hours) (FIG. 5B), and (7 days) (FIG. 5C) after administration of [symbol] saline or Formulation A. FIG. 5D shows the serum peptidoglycan levels in adult female rats.

FIGS. 6A-B compare the concentration of vitamin B12 in ileum in female rat pups on second (FIG. 6A) and third day (FIG. 6B) after treatment with either [symbol] saline or Formulation A. FIGS. 6C-D compare the concentration of vitamin B12 in ileum in male rat pups on second (FIG. 6C) and third day (FIG. 6D) after treatment with either [symbol] saline or Formulation A.

FIGS. 7A-C compare the concentration of various SCFA (cecum acetate shown in FIG. 7A; cecum propionate shown in FIG. 7B; cecum butyrate shown in FIG. 7C) in [symbol] saline or Formulation A-treated female pups 2 days after administration of [symbol] saline or Formulation A. FIGS. 7D-F compare the concentration of various SCFA (cecum acetate shown in FIG. 7D; cecum propionate shown in FIG. 7E; cecum butyrate shown in FIG. 7F) in [symbol] saline or Formulation A-treated male pups 2 days after administration of [symbol] saline or Formulation A.

FIGS. 8A-B compare the distal colon fecal mass in [symbol] saline or Formulation A-treated female rat pups (FIG. 8A) and male rat pups (FIG. 8B) 2 and 3 days after administration of [symbol] saline or Formulation A. FIG. 8C-D compare the fecal water mass in [symbol] saline or Formulation A-treated female pups (FIG. 8C) and male pups (FIG. 8D) 2 and 3 days after administration of [symbol] saline or Formulation A.

FIG. 9A shows the concentration of serum oxytocin in dams after pup withdrawal. FIG. 9B compares the serum oxytocin levels in 16 day old male pups 24 hours after administration of [symbol] saline or Formulation A. FIG. 9C compares the serum oxytocin levels in 20 day old male rat pups 48 hours after administration of [symbol] saline or Formulation A.

FIG. 10 is a graph showing plasma oxytocin levels in female rats following treatment with Formulation B, planktonic L. reuteri (Lr), or saline. Oxytocin levels are shown as an average±standard error mean for the first three days post-treatment (day noted above bars). ANOVA followed by Tukey's multiple comparison was used to compare groups.

FIG. 11A is a graph showing plasma oxytocin levels in female pups following treatment with Formulation B, planktonic L. reuteri (Lr), or saline. FIG. 11B is a graph showing plasma oxytocin levels in male rat pups following treatment with Formulation B, planktonic L. reuteri (Lr), or saline. The values are shown as an average±standard error mean for the first three days post-treatment (day noted above bars). ANOVA followed by Tukey's multiple comparison test was used to compare groups.

FIG. 12 illustrates the experimental study design for [symbol] Lactobacillus reuteri (“L. reuteri”) formulation after administration of prescription grade Azithromycin ( [symbol] Azithromycin).

FIGS. 13A-D show the stomach content dry mass, stomach water content, stomach caloric content, and pH of stomach content in saline-treated and Formulation A-treated female rats, respectively.

FIGS. 14A-B show fecal calprotectin and fecal lactotransferrin levels in saline-treated and Formulation A-treated female rats, respectively. FIGS. 14C-D show fecal IL-22 and fecal IL-6 levels in saline-treated and Formulation A-treated female rats, respectively. The data was collected between days 3 and 4, between days 4 and 5, and between days 5 and 6 after administration of [symbol] Azithromycin.

FIGS. 15A-C show IL-6, IL-10, and IL-22 levels in ileum of saline-treated and Formulation A-treated female rats on the third day after administration of either R Formulation A or [symbol] saline, respectively.

FIG. 16A shows Glucagon-like peptide 2 (GLP-2) levels in proximal colon of saline-treated and Formulation A-treated female rats on third day after administration of either [symbol] Formulation A or [symbol] saline. FIG. 16B compares serum peptidoglycan levels in female rats on third day after administration of [symbol] saline or [symbol] Formulation A.

FIG. 17 shows fecal sIgA levels as measured on days 1, 2, and 3 after administration of either [symbol] Formulation A or [symbol] saline (or days 4, 5, and 6 after administration of [symbol] Azithromycin) in female rats to assess gut protection.

FIGS. 18A-C compare the concentration of various SCFA (fecal acetate shown in FIG. 18A; fecal propionate shown in FIG. 18B; fecal butyrate shown in FIG. 18C) in fecal pellets collected between days 3 and 4, between days 4 and 5, and between days 5 and 6 from [symbol] saline or [symbol] Formulation A-treated female rats.

FIGS. 19A-D compare the concentration of various SCFA (fecal acetate shown in FIG. 19A; fecal propionate shown in FIG. 19B; fecal butyrate shown in FIG. 19C; fecal isovalerate shown in FIG. 19D) in [symbol] saline or [symbol] Formulation A-treated female rats on third day after administration of either [symbol] saline or [symbol] Formulation A.

### DETAILED DESCRIPTION

The present disclosure relates in part to methods for treating or preventing a disease or a disorder using bacterial formulation that comprises a probiotic bacterium, a biocompatible microsphere, and/or a prebiotic.

Unless defined otherwise, all technical and scientific terms used herein have the same meanings as commonly understood by one of ordinary skill in the art to which this disclosure belongs. All technical and patent publications cited herein are incorporated herein by reference in their entirety.

The practice of the present technology will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration and molecular weight, including ranges, are approximations which are varied (+) or (−) by increments of 1.0 or 0.1, as appropriate, or alternatively by a variation of +/−15%, or alternatively 10%, or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a bacterium” includes a plurality of bacteria, including mixtures thereof.

As used in the specification, the term “formulation” may be used interchangeably with “composition”.

The following definitions are included for the purpose of understanding the present subject matter and for constructing the appended patent claims. Abbreviations used herein have their conventional meaning within the chemical and biological arts.

### Definitions

As used herein, the terms “treating,” “treatment” and the like are used herein to mean obtaining a desired pharmacologic and/or physiologic effect. The effect may be prophylactic in terms of completely or partially preventing a disorder or sign or symptom thereof and/or may be therapeutic in terms of a partial or complete cure for a disorder and/or adverse effect attributable to the disorder.

To “prevent” intends to prevent a disorder or effect in vitro or in vivo in a system or subject that is predisposed to the disorder or effect. Examples of such is preventing obesity by achieving satiety in a subject.

A “subject” or “patient” as described herein, refers to any animal at risk for, suffering from or diagnosed for a disease or disorder (for example, autism spectrum disorder, depression, anxiety disorder, or developmental disorder) including, but not limited to, mammals, primates, and humans. In certain embodiments, the subject may be a non-human mammal such as, for example, a cat, a rat, a dog, or a horse. In certain embodiments, the subject is a human subject. In certain embodiments, the subject is a male or female. In some embodiments, the subject is an infant, a child, or an adult. In certain embodiments, subjects may be postpartum women, women who are pregnant, or women who are lactating.

A “prebiotic” intends a nutritional supplement for the probiotic bacterium. Prebiotics are food ingredients, for example, oligosaccharides, that are non-digestible by a subject (e.g., by a mammal such as a human), and that stimulates the growth or activity of one or more beneficial bacteria and/or inhibit the growth or activity of one or more pathogenic bacteria. A prebiotic may selectively stimulate the growth and/or activity of one or a limited number of bacteria in the subject.

A “microsphere” intends a porous and/or semi-permeable biofilm-carrying and/or compound-carrying (e.g., drug-carrying) particulate or granular material within the particular size range recited. As used herein, a microsphere consisting of particles 50 millimeters or less in diameter, and about 1 micron or more (e.g., about 1 to about 100 or alternatively, or alternatively about 1 to about 75 microns, or alternatively about 1 to about 50, or alternatively about 1 to about 25, or alternatively about 1 to about 10 microns, or alternatively about 0.5 to about 200 microns, or alternatively about 0.5 to about 700 microns, or alternatively about 1 to about 600 microns, or alternatively less than about 700 microns, or alternatively less than about 600 microns, or alternatively less than 500 microns, or alternatively less than about 400 microns, or alternatively less than about 300 microns, or alternatively less than about 200 microns, or alternatively less than about 100 microns) in diameter. Non-limiting examples of such include: hollow microspheres that are porous and/or semi-permeable, and can, in some aspects, contain a pharmaceutical or a drug, microcapsules, (in which the excipient forms a skin or shell that surrounds and contains a cargo, such as a drug, a chemical reductant, or absorptive or adsorptive molecules), and microparticles, which are used as a generic term for any particles in the recited size range, whether spherical or not, as those terms are typically used in the art.

A “biodegradable polymer” intends polymers that are biocompatible and can degrade in vivo by bodily processes to products that are readily disposable by the body and should not accumulate in the body.

By “biocompatible”, it is meant that the components of the delivery system will not cause tissue injury or injury to the human biological system. To impart biocompatibility, polymers and excipients that have had history of safe use in humans or with GRAS (Generally Accepted As Safe) status, are preferentially used. By biocompatibility, it is meant that the ingredients and excipients used in the composition will ultimately be “bioabsorbed” or cleared by the body with no adverse effects to the body. For a composition to be biocompatible, and be regarded as non-toxic, it must not cause toxicity to cells. Similarly, the term “bioabsorbable” refers to microspheres made from materials which undergo bioabsorption in vivo over a period of time such that long term accumulation of the material in the patient is avoided. The biocompatible nanoparticle may be bioabsorbed over a period of less than 2 years, less than 1 year, or e.g., less than 6 months. The rate of bioabsorption is related to the size of the particle, the material used, and other factors well recognized by the skilled artisan. A mixture of bioabsorbable, biocompatible materials can be used to form the microspheres in the formulation used in this disclosure.

“Pharmaceutically acceptable carriers” refers to any diluents, excipients or carriers that may be used in the compositions of the disclosure. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like and consistent with conventional pharmaceutical practices.

“Administration” intends the delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration include oral administration, vaginal, enteral, nasal administration (inhalation), injection, topical application and by suppository.

The term “effective amount” refers to a quantity sufficient to achieve a beneficial or desired result or effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of a therapeutic composition, in some embodiments, the effective amount is the amount sufficient to result in a protective response against a pathogen or alternatively to support a healthy state of being. In some embodiments, the amount is sufficient to accomplish one or more of 1) clear pathogen; 2) restore healthy microbiota; 3) modulate the immune system; 4) maintain metabolism and metabolic pathways; 5) reduce caloric intake; and 6) improve social behavior.

In the case of an in vitro or ex vivo applications, in some embodiments, the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment.

The agents and compositions can be used in the manufacture of medicaments and for the treatment of humans and other animals by administration in accordance with conventional procedures, such as an active ingredient in pharmaceutical compositions.

### Compositions

Compositions for use in the disclosed methods may comprise a pharmaceutically acceptable excipient, e.g., a microsphere, a probiotic bacterium (e.g., L. reuteri), and a prebiotic, wherein the prebiotic comprises a nutritional supplementation for the probiotic bacterium. In one aspect, the composition further comprises one or more of: a biofilm, a prebiofilmic, coating on the surface of the microsphere a therapeutic drug or agent, a chemical reductant, a molecule that promotes adsorption, a molecule that supports absorption. A disclosed microsphere may have a solid core, a hollow core, or a porous core, wherein in one aspect, the microsphere encapsulates the prebiotic within the hollow core or a porous core. The microsphere can be biocompatible and/or semi-permeable. In one aspect, the microsphere comprise a biofilm layer or coating on the external surface of the microsphere. In one aspect the microsphere activates the bacteria enhancing persistence and function.

Contemplated biocompatible microspheres may include a material selected from the group of: a biodegradable polymer and/or a non-degradable polymer, and such disclosed microspheres may have a from about 0.5 microns to about 1000 microns. Additional preferred ranges are described herein and incorporated herein by reference. The microspheres can be porous and/or semi-permeable.

Non-limiting examples of biodegradable polymers are selected from one or more of: dextran; dextranomer; e.g. Sephadex (dextran cross-linked with epichlorohydrin), Sephadex G-25; poly(lactic-co-glycolic acid) or PLGA; polycaprolactone or PLC; chitosan; gelatin; acetalated dextran; poly(lactide); poly(glycolide); poly(lactide-co-glycolide); poly(lactic acid); poly(glycolic acid); poly(lactic acid-co-glycolic acid); poly(lactide)/poly(ethylene glycol) copolymers; poly(glycolide)/poly(ethylene glycol) copolymer; poly(lactide-co-glycolide)/poly(ethylene glycol) copolymers; poly(lactic acid)/poly(ethylene glycol) copolymer; poly(glycolic acid)/poly(ethylene glycol) copolymer; poly(lactic acid-co-glycolic acid)/poly(ethylene glycol) copolymer; poly(caprolactone); poly(caprolactone)/poly(ethylene glycol) copolymer; poly(orthoester); poly(phosphazene); poly(hydroxybutyrate); poly(hydroxybutyrate); poly(lactide-co-caprolactone); polycarbonate; polyesteramide; polyanhidride; poly(dioxanone); poly(alkylene alkylate); polyethylene glycol/polyorthoester copolymer; polyurethane; poly(amino acid); polyetherester; polyacetal; polycyanoacrylate; poly(oxyethylene)/poly(oxypropylene) copolymer; Sephadex® copolymers and/or a combination thereof.

Non-limiting examples of non-biodegradable polymers are selected from one or more of poly(ethylene vinyl acetate), poly(vinyl acetate), silicone polymers, polyurethanes, polysaccharides such as a cellulosic polymers and cellulose derivatives, acyl substituted cellulose acetates and derivatives thereof, copolymers of poly(ethylene glycol) and poly(butylene terephthalate), polystyrenes, polyvinyl chloride, polyvinyl fluoride, poly(vinyl imidazole), chlorosulphonated polyolefins, polyethylene oxide, and copolymers and blends thereof.

Contemplated compositions for use in disclosed methods may include a prebiotic for example, one or more of: a water-soluble carbohydrate, inulin, oligosaccharides, oligofructose, fructo-oligosaccharide, galacto-oligosaccharide, glucose, starch, maltose, maltodextrins, polydextrose, amylose, sucrose, fructose, lactose, isomaltulose, polyols, glycerol, carbonate, thiamine, choline, histidine, trehalos, nitrogen, sodium nitrate, ammonium nitrate, beta-glucan, phosphorus, phosphate salts, hydroxyapatite, potassium, potash, sulfur, homopolysaccharide, heteropolysaccharide, cellulose, chitin, vitamins, and combination thereof.

In another aspect, a prebiotic is selected from one or more of trehalose; nitrogen such as in sodium nitrate, ammonium nitrate, phosphorus such in phosphate salts like hydroxyapatite, potassium such as in potash, sulfur, oligosaccharide, homopolysaccharide, heteropolysaccharide, cellulose, chitin, glucose, fructose, sucrose, maltose, starch, polydextrose, amylose, glycerol, carbonate, and combinations thereof. Disclosed herein for example, is a composition that includes maltose and/or L. reuteri.

A composition for use in the disclosed methods may include a probiotic bacterium selected to provide one or more of supporting anti-bacterial immunity, enhancing or supporting a healthy state in the subject, enhancing or supporting the gastrointestinal barrier, or antagonizing disease-related bacterial infections. In another aspect, the probiotic bacterium is selected to prevent pathogen colonization and/or limit and/or clear the pathogen, and/or limit excessive inflammatory responses by down-regulating cytokine and chemokine production. In certain embodiments, the probiotic bacterium is selected to induce hormones that have roles in social bonding, energy metabolism, wound healing contributing to good physical, mental, and social health. Non-limiting examples of the probiotic bacterium is one or more of L. acidophilus, L. crispatus, L. gasseri, group L. delbrueckii, L. salivarius, L. casei, L. paracasei, L. plantarum, L. rhamnosus, L. reuteri, L. brevis, L. buchneri, L. fermentum, L. rhamnosus, B. adolescentis, B. angulation, B. bifidum, B. breve, B. catenulatum, B. infantis, B. lactis, B. longum, B. pseudocatenulatum, S. thermophiles, Pseudomonas fluorescens, P. protegens, P. brassicacearum, P. aeruginosa; Azospirillum. brabrasilense, A. hpferum, A. halopraeferens, A. irakense; Acetobacter diazotrophicus; Herbaspirillum seropedicae; Bacillus subtilis, Pseudomonas stutzeri, fluorescens, P. putida, P. cepacian, P. vesicularis, P. paucimobilis; Bacillus cereus, B. thuringiensis, B. sphaericus; Shewanella oneidensis; Geobacter bemidjiensis, G. metallireducens, G. sulfurreducens, G. uraniireducens, G. lovleyi; Serratia marcescens, Desulfovibrio vulgaris, D. desulfuricans, Dechloromonas aromatic, Deinococcus radiodurans, Methylibium petroleiphilum, Alcanivorax borkumensis, Archaeglobus fulgidus, Haloferax sp., Halobacterium sp., and combinations thereof. In certain embodiments, a composition for use in disclosed methods of treatment provided includes L. reuteri (ATCC® 23272™).

Disclosed compositions, may include further agents, e.g., an agent selective against a pathogen that may compete with the probiotic organism. Such complimentary agents can be in the core, on the surface of the microsphere in the composition containing the microspheres. Non-limiting examples of such include chemical reductants; molecules and/or surfaces that promote adsorption (in core or on surface of microsphere); molecules and/or surfaces that promote absorption (in core or on surface of microsphere). In one aspect, the chemical reductants and molecules and/or surfaces that promote absorption are coated on the surface of the microsphere.

For example, a disclosed composition may include colonies of activated bacteria that may form a biofilm layer on the external surface of the microparticle, e.g., that includes L. reuteri. Such a layer can be from about 0.5 micron to about 1 millimeter in depth, and ranges in between, e.g., about 1 micron to about 500 microns, about 1 micron to about 250 microns, about 1 micron to about 200 microns, about 1 micron to about 100 microns, about 1 micron to about 50 microns, about 1 micron to about 40 microns, about 1 micron to about 30 microns, about 2 micron to about 100 microns, about 2 microns to about 50 microns, about 2 microns to about 40 microns, about 2 microns to about 30 microns, about 3 microns to about 100 microns, about 3 microns to about 50 microns, about 3 microns to about 40 microns, about 3 microns to about 30 microns, about 5 microns to about 100 microns, about 5 microns to about 50 microns, about 5 microns to about 40 microns, and about 5 microns to about 30 microns. In other embodiments, a contemplated composition includes a scaffold or matrix for with the ability to support the activation of bacteria (e.g. L. reuteri) upon administration to a subject or an environment to be treated.

As contemplated herein, compositions for use in disclosed methods may comprise one or a plurality of microsphere compositions in combination with a carrier, e.g., a pharmaceutically acceptable carrier or a biocompatible scaffold. Non-limiting examples of pharmaceutically acceptable carriers include diluents, excipients or carriers that may be used in the compositions of the disclosure. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Disclosed compositions can be formulated into dosage forms of the biofilm-generative, activated probiotic bacterium, e.g., or provide from an effective amount of the microsphere composition for the end use, e.g., from about 1×10⁵ to 1×10¹¹ CFU/ml, or alternatively from about 1×10⁵ to about 1×10¹¹ CFU/ml, or about 1×10⁵ to about 1×10⁹ CFU/ml, or about 1×10⁶ to about 1×10¹¹ CFU/ml, or about 1×10⁶ to about 1×10⁹ CFU/ml, or about 1×10⁷ to about 1×10¹¹ CFU/ml, or about 1×10⁷ to about 1×10¹⁰ CFU/ml, or about 1×10⁷ to about 1×10⁹ CFU/ml, or about 1×10⁸ CFU/ml.

In certain embodiments, the bacterial formulation (e.g., L. reuteri formulation) can be administered at about 6, 12, 18, 24, 36, 48, and 72 hours, or can be administered in a single dose for treatment. In certain embodiments, the bacterial formulation (e.g., L. reuteri formulation) can be administered orally, vaginally, topically, by inhalation, intravenously, intramuscularly, or by suppository. They can be administered in any suitable formulation.

The compositions can be formulated or processed for ease of administration, storage and application, e.g., frozen, lyophilized, suspended (suspension formulation) or powdered; and processed as a suppository, tablet, solution, suspensions, pills, capsules, sustained release formulation.

An effective amount of therapeutic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

In certain embodiments, the present disclosure provides methods of treatment by administration of a therapeutically effective amount of a bacterial formulation (e.g.; L. reuteri formulation). In certain embodiments, bacterial formulation comprises Lactobacillus reuteri, a biocompatible microsphere, and/or a prebiotic.

### Methods

Provided herein are methods for treating or preventing a disease or disorder suitably treated by a bacterial formulation in a subject in need thereof. The method comprises administering to the subject an effective amount of the bacterial formulation as disclosed herein, having the components selected for the particular therapy. The present disclosure provides, in an embodiment, methods of inducing oxytocin by administration of a therapeutically effective amount of a bacterial formulation (e.g.; L. reuteri formulation). In certain embodiments, oxytocin is induced in a subject by administration of therapeutically effective amount of a composition comprising Lactobacillus reuteri, a biocompatible microsphere, and/or a prebiotic. In certain embodiments, administration of a therapeutically effective amount of a bacterial formulation (e.g.; L. reuteri formulation) activates the oxytocin receptor.

In certain embodiments, the present disclosure, in an embodiment, provides methods of treating, ameliorating and/or preventing autism spectrum disorder, including but not limited to, autistic disorder, Asperger syndrome, Heller's syndrome, Rett syndrome, and Pervasive developmental disorder, not otherwise specified (PDD-NOS) in a subject by administration of pharmaceutically acceptable composition of bacterial formulation (e.g., L. reuteri formulation) to the subject.

Also provided herein are methods for treating or preventing a disease or disorder (e.g., gastrointestinal inflammation) due to administration of an antibiotic regimen suitably treated by a bacterial formulation in a subject in need thereof. The method comprises administering to the subject an effective amount of the bacterial formulation as disclosed herein, having the components selected for the particular therapy. Non-limiting examples of diseases include gastrointestinal disorders, such as gastrointestinal inflammation, or any of chronic and/or recurrent disease that is caused by the use of antibiotics and results in pathogenic bacteria displacing healthy bacteria, disease or disorders involving intestinal dysmobility, or antagonizing disease-related bacterial infection.

The present disclosure provides, in an embodiment, methods of treating gastrointestinal inflammation in a patient due to administration of an antibiotic regimen (e.g., azithromycin) by administration of a therapeutically effective amount of a bacterial formulation (e.g., L. reuteri formulation). In certain embodiments, gastrointestinal inflammation is reduced or mitigated in a subject by administration of therapeutically effective amount of a composition comprising Lactobacillus reuteri, a biocompatible microsphere, and/or a prebiotic.

In certain embodiments, the present disclosure provides a method of treating a patient having or expected to have gastrointestinal inflammation associated with administration of an oral antibiotic regimen by administering to the patient in need a therapeutically effective amount of a composition comprising Lactobacillus reuteri and a pharmaceutically acceptable carrier. In certain embodiments, gastrointestinal inflammation is significantly reduced by administration of therapeutically effective amount of a composition comprising Lactobacillus reuteri, a biocompatible microsphere, and/or a prebiotic. Such oral antibiotic regimens may include oral antibiotics administration of the following general classes for example, sulfa drugs; folic acid analogs; beta-lactams, including penicillins and cephalosporins; tetracyclines; macrolides; lincosamides; streptogramins; quinolones, including fluoroquinolones; polypeptides such as polymixins; aminocyclitols; glycopeptides; oxazolidinones; and the like. “Antibiotic” means antibiotics, antibacterials, antimicrobials, antiinfectives, and the like.

Exemplary antibiotics include amoxicillin, the combination of amoxicillin and potassium clavulanate, ampicillin, the combination of ampicillin and sulbactam, atovaquone, azithromycin, carbenicillin, cefaclor, cefdinir, cefonicid, ceftibuten, cefotetan, cefpodoxime, ceftriaxone, cefuroxime, cephalexin, cephalothin, cephamycin, chlortetracycline, ciprofloxacin, clindamycin, clarithromycin, cycloserine, dalfopristin, dicloxacillin, doxycycline, erythomycin, levofloxacin, linezolid, moxifloxacin, mupirocin, oxytetracycline, penicillin, rifampin, quinupristin, the combination of dalfopristin and quinupristin, spectinomycin, sulfadiazine, sulfamethoxazole, sulfametrole, sulfamoxole, sulfalene, sulfanilamide, tetracycline, trimethoprim, the combination of trimethoprim and sulfamethoxazole, vancomycin, a combination comprising at least one of the foregoing, and the like. In certain embodiments, the antibiotic regimen comprises azithromycin

In certain embodiments, the present disclosure provides a method of treating a patient for a gastrointestinal disorder, comprising administering a composition comprising Lactobacillus reuteri, a biocompatible microsphere, and/or a prebiotic. In certain embodiments, the gastrointestinal disorder is one or more of constipation, abdominal pain, flatulence, and diarrhea.

In certain embodiments, the present disclosure provides a method of treating a patient for a gastrointestinal disorder, wherein the patient is on the autism spectrum or is autistic.

Provided herein methods for substantially preventing or decreasing an oral antibiotic-associated adverse effect in a subject in need thereof, using a therapeutically effective amount of a composition comprising Lactobacillus reuteri, a biocompatible microsphere, and/or a prebiotic.

In certain embodiments, the bacterial formulations (e.g., L. reuteri formulation) described in the present disclosure may be used for preventing and/or ameliorating and/or treating adverse gastrointestinal (GI) side effects (e.g., from another medication that has a GI side effect, e.g., antibiotic administration) including but not limited to pseudomembranous colitis (inflammation of the large intestine due to an overgrowth of Clostridium difficile), nausea, cramping, diarrhea and vomiting. These are some side effects associated with antibiotics. However, the effects of the formulation employed in the context of the disclosure on these conditions may be mediated in whole or in part via an effect on body weight, or may be independent thereof. In certain embodiments, disclosed bacterial formulations (e.g., L. reuteri formulation) may be used as pharmaceutical agents for preventing the aforementioned disorders and/or diseases. In some embodiments, the administration causes one or more of: a reduction in or elimination of one or more symptoms of the condition, prevention of increased severity of one or more symptoms of the condition, and/or reduction, prevention, or elimination of further diseases or conditions.

In certain embodiments, the administration of pharmaceutically acceptable composition of bacterial formulation (e.g., L. reuteri formulation) described in the present disclosure causes a reduction of gastrointestinal inflammation in the subject. In certain embodiments, the administering causes at least a 5% reduction in the patient's gastrointestinal inflammation (e.g., at least 7%, 10%, 20%, 30%, 50%, 75% reduction or more).

In certain embodiments, the bacterial composition (e.g., L. reuteri formulation) is administered before administration of the antibiotic regimen, during the administration of the antibiotic regimen, and/or after the antibiotic regimen.

The present disclosure provides, in an embodiment, methods of treating depression or an anxiety disorder in a subject by administration of pharmaceutically acceptable composition of bacterial formulation (e.g., L. reuteri formulation) to the subject. In certain embodiments, depression or an anxiety disorder includes clinical depression, postnatal or postpartum depression, postpartum obsessive-compulsive disorder, post-traumatic stress disorder, e.g., postpartum post-traumatic stress disorder, postpartum bipolar disorder, atypical depression, melancholic depression, Psychotic Major Depression (PMD), catatonic depression, Seasonal Affective Disorder (SAD), dysthymia, double depression, Depressive Personality Disorder (DPD), Recurrent Brief Depression (RBD), minor depressive disorder, bipolar disorder or manic depressive disorder, treatment-resistant depression, refractory depression, suicidality, suicidal ideation, and suicidal behavior. In some embodiments, the subject is suffering from an anxiety disorder. In some embodiments, the subject is suffering from depression. In some embodiments, the subject is suffering from postnatal or postpartum depression.

The present disclosure, in an embodiment, provides methods of treating a developmental disorder which is associated with preterm birth in a preterm infant by administration of pharmaceutically acceptable composition of bacterial formulation (e.g., L. reuteri formulation) to the preterm infant. In certain embodiments, the bacterial formulation used for the treatment comprises a therapeutically effective amount of a composition comprising Lactobacillus reuteri and a pharmaceutically acceptable carrier.

In certain embodiments, the present disclosure provides methods of treatment of developmental disorders which are associated with preterm birth, including but not limited to, bronchopulmonary dysplasia (BPD); intelligence deficits, cerebral palsy, white matter disease; and retinopathy of prematurity.

In certain embodiments, the present disclosure provides a method of inducing oxytocin in a preterm infant in need by administering to the preterm infant a therapeutically effective amount of a composition comprising Lactobacillus reuteri and a pharmaceutically acceptable carrier. In certain embodiments, oxytocin is induced in a preterm infant by administration of therapeutically effective amount of a composition comprising Lactobacillus reuteri, a biocompatible microsphere, and/or a prebiotic.

In certain embodiments, administration of at least one daily dose of the bacterial formulation (e.g.; L. reuteri formulation) to the subject results in an upregulation in oxytocin levels. In certain embodiments, oxytocin is upregulated by at least 5% (e.g., at least 5%, 10%, 20%, 30%, 50%, 75% upregulation or more) in the subject after administration.

In certain embodiments, the present disclosure provides a method of enhancing lactation in a subject in need, the method comprising administering to the subject a therapeutically effective amount of a composition comprising Lactobacillus reuteri, a biocompatible microsphere, and/or a prebiotic. In certain embodiments, enhancement of lactation after administration of bacterial formulation (e.g., L. reuteri formulation) of the present disclosure is due to increased levels of oxytocin which is known to be involved in milk let down and secretion (see Pang, W. W. et al.; (2007); J Mammary Gland Biol Neoplasia 12, 211-221).

In certain embodiments, administration of bacterial formulation (e.g., L. reuteri formulation) of the present disclosure increases oxytocin levels (e.g., about 10%, about 20%, about 30%, about 40%, about 50%, about 60%, about 70%, about 80%, about 90%, about 100%, about 120%, about 140%, about 160%, about 180%, about 200%, about 220%, about 240%, about 260%, about 280%, or about 300%). In certain embodiments, a single dose of bacterial formulation (e.g., L. reuteri formulation) elevates oxytocin levels for hours (e.g., about 6 hours, about 12 hours, about 18 hours, about 24 hours, about 36 hours, about 48 hours, about 72 hours, or about 96 hours). In certain embodiments, multiple dose of bacterial formulation (e.g., L. reuteri formulation) elevates oxytocin levels for hours (e.g., about 6 hours, about 12 hours, about 18 hours, about 24 hours, about 36 hours, about 48 hours, about 72 hours, or about 96 hours). In certain embodiments, administration of bacterial formulation (e.g., L. reuteri formulation) of the present disclosure increases prolactin levels (levels (e.g., about 10%, about 20%, about 30%, about 40%, about 50%, about 60%, about 70%, about 80%, about 90%, about 100%, about 120%, about 140%, about 160%, about 180%, about 200%, about 220%, about 240%, about 260%, about 280%, or about 300%).

In some embodiments, a single dose of bacterial formulation (e.g.; L. reuteri formulation) is administered to a subject. In other embodiments, multiple doses are administered over two or more time points, separated by hours, days, weeks (e.g., after about 6 hours, about 12 hours, about 18 hours, about 24 hours, about 36 hours, about 48 hours, about 72 hours). In some embodiments, bacterial formulation (e.g.; L. reuteri formulation) is administered over a long period of time (e.g., chronically), for example, for a period of months or years (e.g., 1, 2, 3, 4, 5, 6, 7, 8, 9, 10, 11, 12, or more months or years). In certain embodiments, bacterial formulation (e.g.; L. reuteri formulation) may be taken on a regular scheduled basis (e.g., daily, weekly, etc.) for the duration of the extended period.

In some embodiments, the composition of the method is administered to provide from about 1×10⁷ to about 1×10¹⁰ CFU/ml (e.g., about 1.5×10⁷, about 2×10⁷, about 2.5×10⁷, about 3×10⁷, about 3.5×10⁷, about 4×10⁷, about 4.5×10⁷, about 5×10⁷, about 5.5×10⁷, about 6×10⁷, about 6.5×10⁷ about 7×10⁷, about 7.5×10⁷, about 8×10⁷, about 8.5×10⁷, about 9×10⁷, about 9.5×10⁷, about 1×10⁸, about 1.5×10⁸, about 2×10⁸, about 2.5×10⁸, about 3×10⁸, about 3.5×10⁸, about 4×10⁸ about 4.5×10⁸, about 5×10⁸, about 5.5×10⁸, about 6×10⁸, about 6.5×10⁸, about 7×10⁸ about 7.5×10⁸, about 8×10⁸, about 8.5×10⁸, about 9×10⁸, about 9.5×10⁸, 1×10⁹, about 1.5×10⁹, about 2×10⁹, about 2.5×10⁹, about 3×10⁹, about 3.5×10⁹, about 4×10⁹, about 4.5×10⁹, about 5×10⁹, about 5.5×10⁹, about 6×10⁹, about 6.5×10⁹, about 7×10⁹, about 7.5×10⁹ about 8×10⁹, about 8.5×10⁹, 9×10⁹, or about 9.5×10⁹) of the activated, biofilm-generating probiotic bacterium.

In certain embodiments, a disclosed bacterial formulation (e.g., L. reuteri formulation) employed in the context of the disclosure may be administered as part of a combination therapy with at least one other agent for treatment of diseases or disorders discussed in the disclosure.

The disclosure now being generally described, it will be more readily understood by reference to the following examples which are included merely for purposes of illustration of certain aspects and embodiments of the present disclosure, and are not intended to limit the disclosure in any way.

### EXAMPLES

The following examples are merely illustrative and are not intended to limit the scope or content of the disclosure in any way.

### Example 1—Formulation of Lactobacillus reuteri

The formulation is provided as a powder for reconstitution into a liquid, administered orally. Each mL of Formulation A contains 1.9×10⁹ colony forming units of L. reuteri and 18.5 mg Sephadex in 74 mM maltose. The dose is 10 mL of the formulation, once daily, for a daily dose of 1.9×10¹⁰ colony forming units of L. reuteri and 185 mg Sephadex in 74 mM maltose.

### Example 2—Study of Lactobacillus reuteri Formulation

A study of L. reuteri formulation was performed. The preclinical safety study design for a L. reuteri formulation includes 60 mg dextranomer microparticle (cross-linked dextran with epicholorhydine) loaded with 24 μmole prebiotic maltose, and 6×10⁹ CFU L. reuteri (ATCC 23272). [Formulation A]. Briefly, naturally delivered neonatal rats were divided into the following treatment groups: (1) prescription grade saline (R saline) and (2) Formulation A. Each treatment group consisted of 24 male and 24 female pups. On day 15, each pup received a single enteral dose of: [symbol] saline or Formulation A. The pups remained with their mothers (dams) throughout the study. The pups breast fed for nutrition. As the pups approached day 21, only Formulation A-treated pup began to consume chow that was placed for the mothers. Pups (N=8 (8 males and 8 females) from each treatment group) were euthanized 24 hours, 48 hours, and 7 days after administration. Histological studies were performed on esophagus, stomach, duodenum, jejunum, ileum, cecum, proximal colon, distal colon, rectum, lungs, kidney, and liver. Histopathology of the entire GI tract in both [symbol] saline- and Formulation A-treated pups appeared normal. Light microscopy imaging indicates that microspheres were cleared intact in feces by 48 hours after oral gavage. Preclinical toxicology studies demonstrate that Formulation A formulation is safe. Macroscopic evaluation of the rat pups show that pups treated with Formulation A consume chow earlier than saline-treated pups, while calories are equivalent on days 17 and 21. On day 17, even though pups consumed less mass with a greater caloric density, their calories are equal to day 21 when there is greater food intake (total mass) at a lower caloric density. This suggests an established hypothalamic set point regulating energy balance. These photos illustrate pups that were supplementing breast feeding with solid food.

### Example 3—Spontaneous Weaning: Effect of Administering L. reuteri Formulation to Neonatal Rats

A weaning study design for neonatal rats as follows. Briefly, naturally delivered pups were divided into the following treatment groups: (1) prescription grade saline (R saline) and (2) Formulation A. Each treatment group consisted of 20 male and 20 female pups. On day 18, each pup received a single enteral dose of: [symbol] saline or Formulation A. Pups (N=10 (10 males and 10 females) from each treatment group) were euthanized 48 hours, and 72 hours after administration. Trunk blood, stomach, jejunum, ileum, cecum, proximal colon, and distal colon were harvested on day 20 and 21. Additionally, body composition, thymus mass, and spleen mass were analyzed for each pup.

### Effect on Caloric Intake

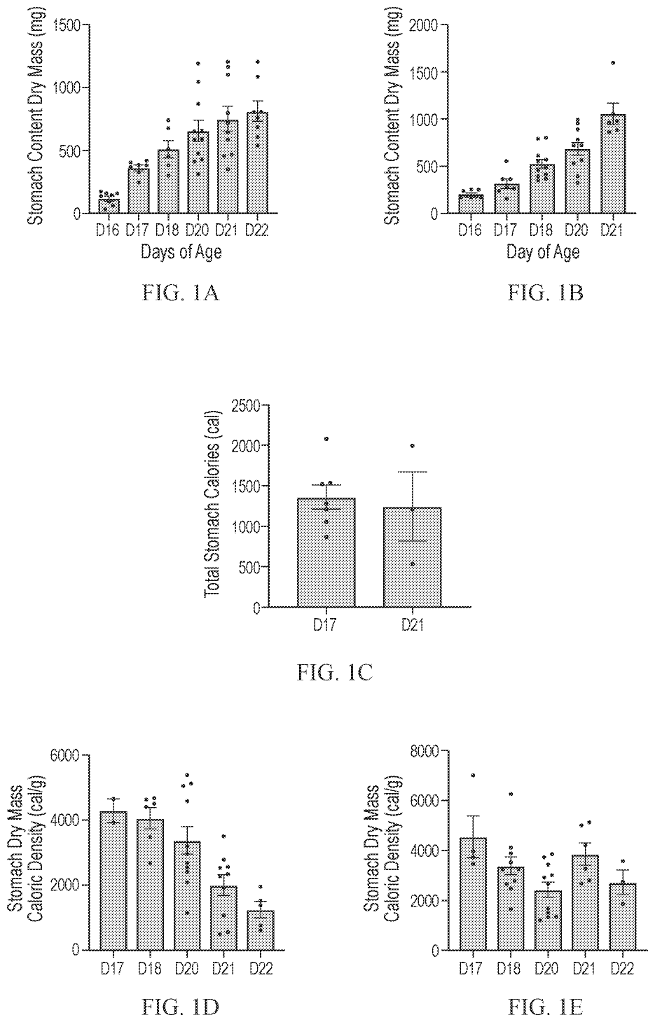

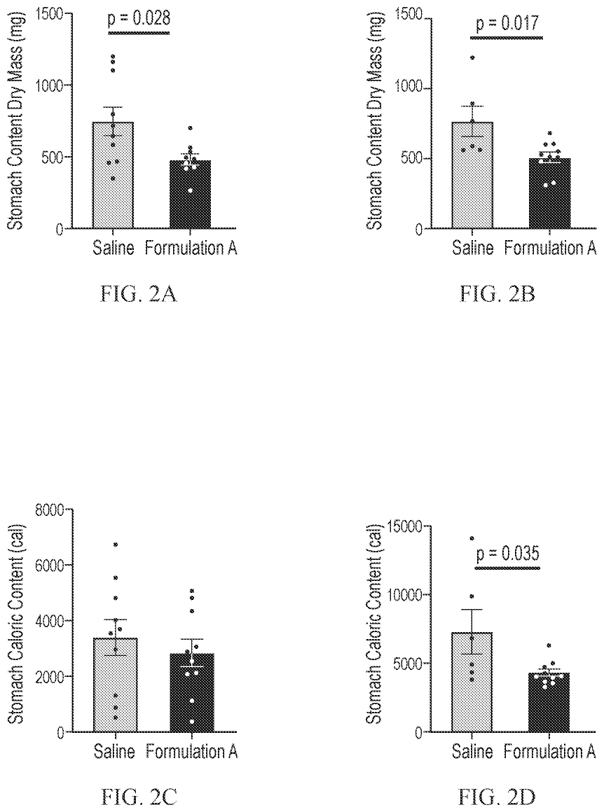

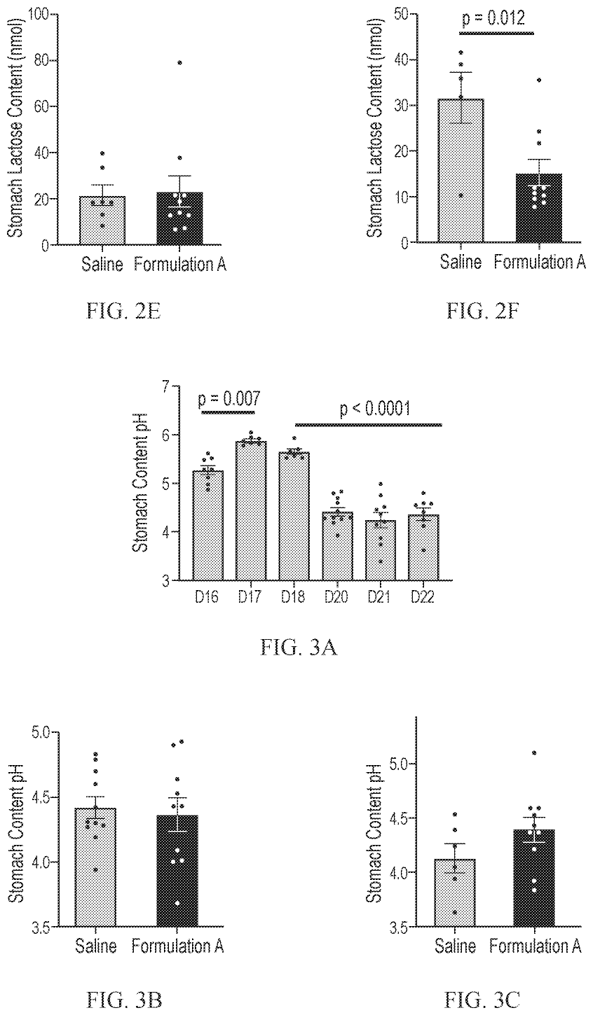

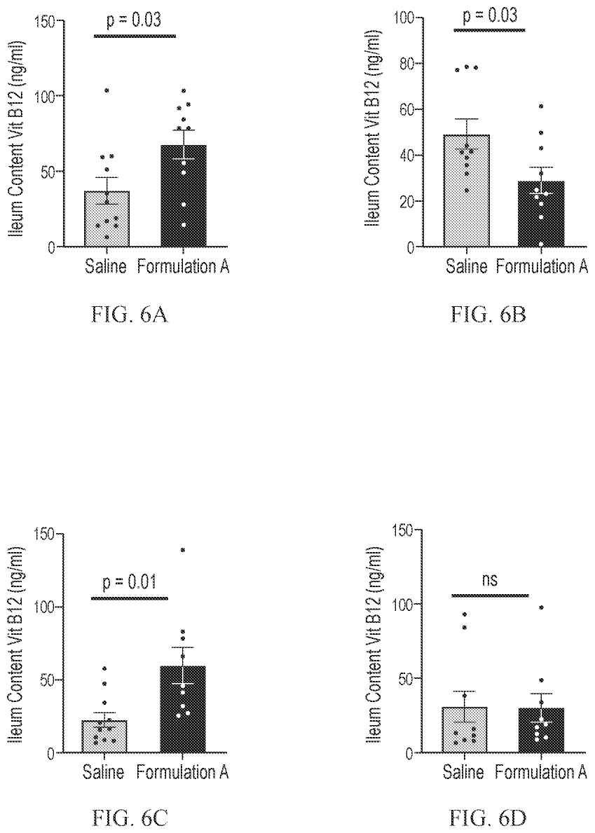

FIGS. 1A-B show the stomach content dry mass in saline-treated female and male pups, respectively. FIG. 1C shows total stomach calories in day 17 (D17) and day 21 (D21) male pups. Total stomach calories were same in female pups. Total calories were maintained with the transition from milk to chow. FIGS. 1A-C show that the calories are equivalent on D17 and D21. On day 17, even though pups consumed less mass with a greater caloric density, their calories are equal to day 21 when there is greater food intake (total mass) at a lower caloric density. This suggests an established hypothalamic set point regulating energy balance. FIGS. 1D-E show the caloric density in saline-treated female and male pups, respectively. FIGS. 1A-E show that the mass of stomach content increases while caloric density decreases in saline-treated pups as they approach weaning. FIGS. 2A-B compare the stomach content dry mass in Formulation A-treated and [symbol] saline-treated pups (data from D21 female and D21 male pups are shown in FIGS. 2A and 2B, respectively). FIGS. 2C-D compare the stomach caloric content in Formulation A-treated and [symbol] saline-treated pups (data from D21 female and D21 male pups are shown in FIGS. 6C and 6D, respectively). FIGS. 2E-F compare the stomach lactose content in Formulation A-treated and [symbol] saline-treated pups (data from D21 female and D21 male pups are shown in FIGS. 2E and 2F, respectively). FIGS. 2A-F show that despite an established caloric intake set point, Formulation A decreases food intake. FIGS. 2A-F also suggest that Formulation A treatment may hasten the switch from milk to chow earlier in males.

### Effect on Stomach Acid

Intragastric pH measurements were taken to test the effect of L. reuteri formulation on stomach acid. Briefly, the animals were euthanized, and the stomach content was removed for the assays. Data for day 16-19, and 22 pups were obtained from safety study described in Example 2. Data described in FIG. 3A is from rat pups from saline treatment or no treatment. FIG. 3A shows the stomach content pH in saline-treated female pups. FIGS. 3B-C compare the stomach content pH in Formulation A-treated and [symbol] saline-treated pups (data from D20 female and D21 male pups are shown in FIGS. 3B and 3C, respectively). FIGS. 3A-C show that production of stomach acid increases after 18 days of age. FIGS. 3A-C also show that administration of Formulation A does not promote weaning by increasing stomach acid production.

### Effect on Jejunum Protein Concentration

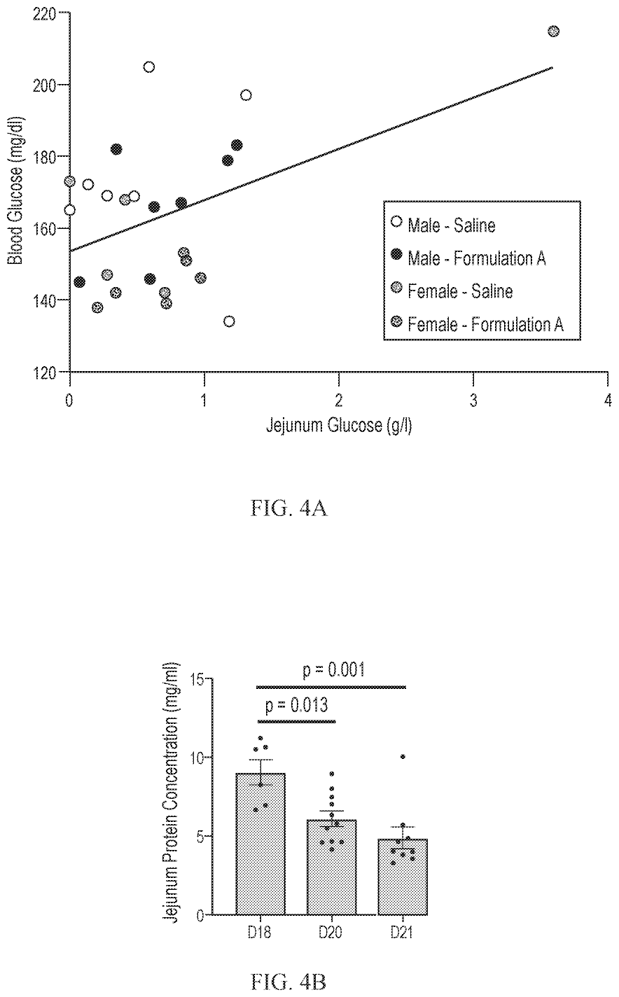

Jejunum is specialized for absorption of nutrients. Briefly, the jejunum was isolated from euthanized pups. The contents of the jejunum were harvested into a vial and were stored at −20° C. until assayed. FIG. 4A shows blood glucose and jejunum glucose levels. FIG. 4B shows jejunum protein concentration in female rat pups. FIG. 4A also shows that blood glucose is dependent on jejunum glucose concentration. FIG. 4A shows that function of intestinal glucose transporters (SGLT-1, GLUT-2) and fructose transporters (GLUTS) are active in rat pups from day 16. FIG. 4B shows that jejunum protein concentration is dependent on production of stomach acid and as a result decreases after 18 days of age.

### Effect on Serum Peptidoglycan Concentration

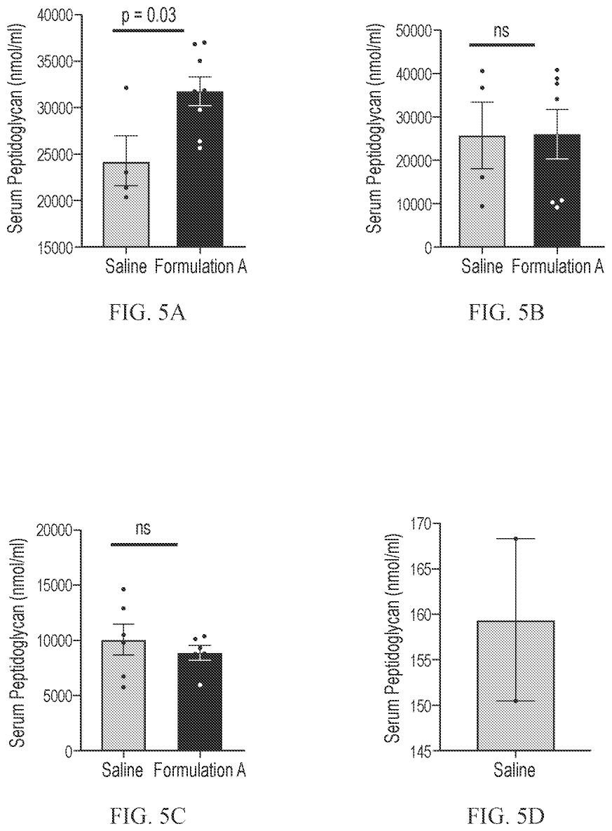

Serum peptidoglycan levels were measured in Formulation A-treated female pups. Briefly, the pups were euthanized by decapitation and trunk blood was collected into tubes and allowed to clot when placed in ice. Serum was separated by centrifugation and stored at −20° C. until assayed for peptidoglycan by enzyme-linked immunosorbent assay (ELISA). FIGS. 5A-C compare the serum peptidoglycan levels in 16 day old female pups (24 hours) (FIG. 5A), 17 day old pups (48 hours) (FIG. 5B), and 21 day old pups (7 days) (FIG. 5C) after administration of [symbol] saline or Formulation A. FIG. 5D shows the serum peptidoglycan levels in adult female rats.

### Effect on Vitamin B12 Production

Ileum vitamin B12 content is measured on the second and third day after treatment with either Formulation A or [symbol] saline in female and male pups. Briefly, the ileum was isolated from euthanized pups. The contents of the ileum were harvested into a vial and were stored at −20° C. until assayed. Vitamin B12 was measured by ELISA. FIGS. 6A-B compare the concentration of vitamin B12 in ileum in female rat pups on second (FIG. 6A) and third day (FIG. 6B) after treatment with either [symbol] saline or Formulation A. FIGS. 6C-D compare the concentration of vitamin B12 in ileum in male rat pups on second (FIG. 6C) and third day (FIG. 6D) after treatment with either [symbol] saline or Formulation A. FIGS. 6A and 6C show that administration of Formulation A increases vitamin B12 concentration in ileum of rat pups (males and females). Vitamin B12 is synthesized by L. reuteri in the ileum. FIG. 6B shows that there is a decrease in vitamin B12 concentration in ileum in of Formulation A-treated female rat pups. FIG. 6D show that there is no statistical difference in vitamin B12 concentration in [symbol] saline or Formulation A-treated male pups. These graphs show that vitamin B12 is cleared from ileum by day 3. Vitamin B12 is also used as a marker of reuterin, a potent antimicrobial compound produced by L. reuteri.

### Effect on Short Chain Fatty Acids (SCFA)

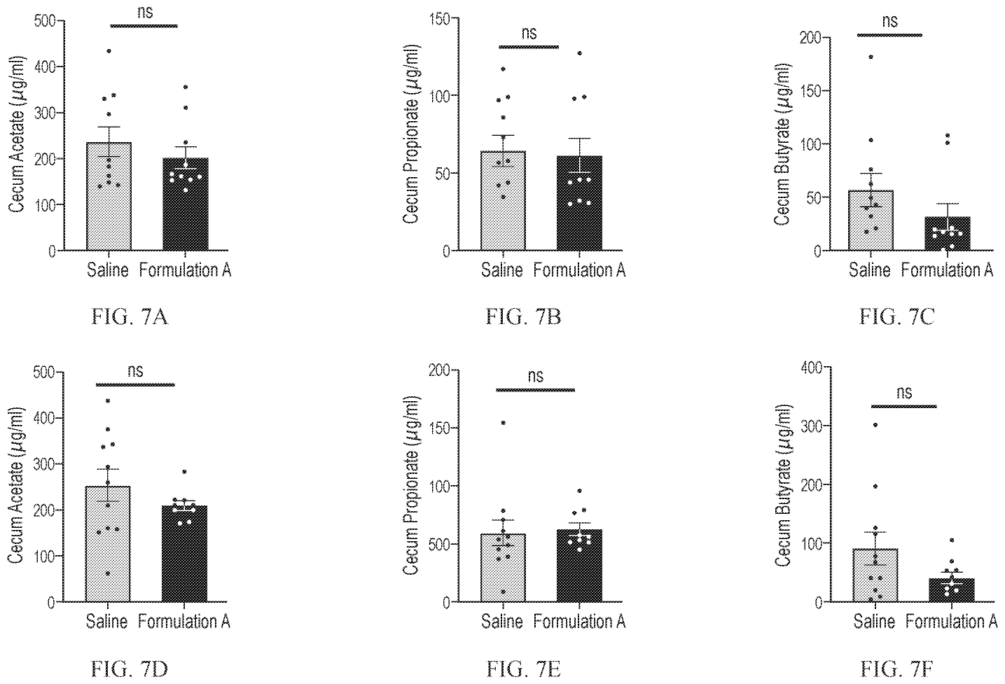

Measurement of SCFA (acetate, propionate, and butyrate) were performed by Gas chromatography-mass spectrometry (GC/MS). Briefly, the cecum was isolated from euthanized pups. The contents of the cecum were harvested into a vial and were stored at −20° C. until assayed. The samples were sonicated in 0.5% phosphoric acid (200 mg/ml), centrifuged to remove solid, unextracted material and filtered through 0.6 μm filter to remove any large particles. Those samples were injected (1 μl) into a GC/MS with a Stabilwax-DA column. A standard solution of 6 short chain fatty acids was used to calibrate the samples. FIGS. 7A-C compare the concentration of various SCFA (cecum acetate shown in FIG. 7A; cecum propionate shown in FIG. 7B; cecum butyrate shown in FIG. 7C) in [symbol] saline or Formulation A-treated female pups 2 days after administration of [symbol] saline or Formulation A. FIGS. 7D-F compare the concentration of various SCFA (cecum acetate shown in FIG. 7D; cecum propionate shown in FIG. 7E; cecum butyrate shown in FIG. 7F) in [symbol] saline or Formulation A-treated male pups 2 days after administration of [symbol] saline or Formulation A. FIGS. 7A-F show that administration of Formulation A does not alter SCFA production in the cecum in rat pups (males and females), and as a result the weaning effects are not via SCFA production.

### Effect on Fecal Output

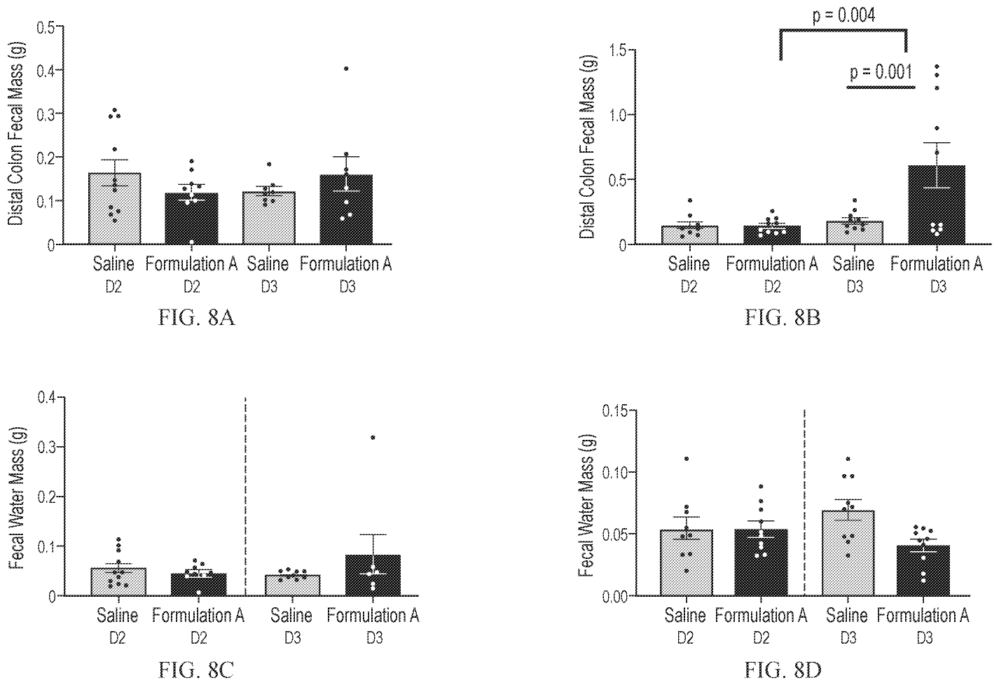

The distal colon was isolated from euthanized pups. Fecal pellets in the distal colon were harvested into a vial and were stored at −20° C. until assayed. To measure dry mass and water mass, the fecal pellets were lyophilized to remove water. The difference between wet mass and dry mass was water mass. FIGS. 8A-B compare the distal colon fecal mass in [symbol] saline or Formulation A-treated female pups (FIG. 8A) and male pups (FIG. 8B) 2 and 3 days after administration of [symbol] saline or Formulation A. FIG. 8B shows an increase in fecal output in Formulation A-treated male pups. The data obtained from FIG. 8B also shows that Formulation A can be used to treat irregularity. FIG. 8C-D compare the fecal water mass in [symbol] saline or Formulation A-treated female pups (FIG. 8C) and male pups (FIG. 8D) 2 and 3 days after administration of [symbol] saline or Formulation A. FIGS. 8C-D show that administration of Formulation A does not cause diarrhea or constipation in males or females rat pups.

The data presented in Example 3 shows that Formulation A promotes weaning and satiety at the hypothalamic level rather than alter gut function.

### Example 4—Effect of Administering L. reuteri Formulation to Adult Rats

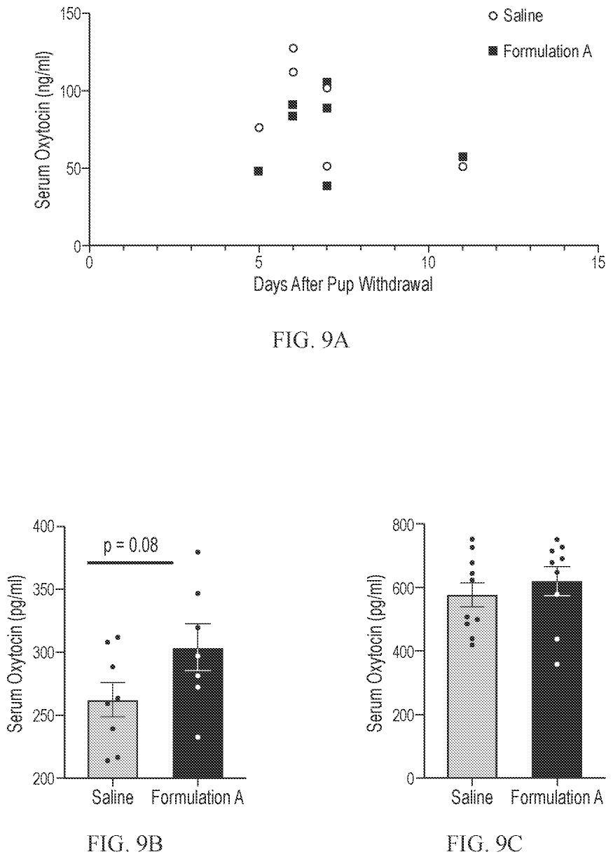

FIG. 9A shows the concentration of serum oxytocin in dams after pup withdrawal. The graph shows that serum oxytocin levels remain elevated for about 11 days after withdrawal of their suckling pups in Formulation A-treated adult female dams. The data shows that elevated oxytocin levels observed during breast feeding are reduced after about 11 days after the dam's pups have been withdrawn. These results show that Formulation A might modulate postpartum depression in female rats. FIG. 9B compare the serum oxytocin levels in 16 day old male pups 24 hours after administration of [symbol] saline or Formulation A. FIG. 9C compare the serum oxytocin levels in 20 day old male pups 48 hours after administration of [symbol] saline or Formulation A. FIGS. 9B-C show that serum oxytocin levels increase in Formulation A-treated male pups after 24 hours of administration and indicating that elevated oxytocin levels might help to modulate food intake in childhood, as well as social and exploratory behavior. Data shown in FIGS. 9B-C was after one dose of Formulation A. Multiple doses of Formulation A might assist in maintaining elevated oxytocin levels.

### Example 5—Activation of L. reuteri with Dextranomer Microparticle (DM)

L. reuteri formulation comprising maltose and DM illustrates enhanced bioactivity. In vitro activation mimics activation in the gastrointestinal tract when the microbe binds to glycans of mucin. DM is a biocompatible porous semi-permeable solid particle that contains dextran, which are similar to O-linked glycans of mucin. L. reuteri strain of the present disclosure expresses the extracellular glucosyltransferase (GTF) enzyme. The in vitro activation process is a simulation of the biological changes induced when L. reuteri comes in contact with the glycans of mucin in the ileum and binds. Thus, activation provides an advantage over the unactivated L. reuteri to better initiate colonization.

### Example 6—Effect of Administering L. reuteri Formulation on Plasma Oxytocin Levels

L. reuteri (ATCC23272 strain) was supplied in vials containing lyophilized L. reuteri at 1.7×10¹¹ CFU/g. 1 mL of sterile saline was added to the lyophilized bacteria and allowed to rehydrate for 5 min at room temperature and then added to the DM-maltose slurry. The DM-maltose slurry was prepared by weighing autoclaved dry Sephadex G25 Superfine microspheres (DM) and added to filter-sterilized 1M maltose solution (25% w/v suspension). In order to initiate the activation step, mixture of L. reuteri and DM-maltose slurry was incubated together at room temperature for 60 minutes prior to use. The final dose per administration of L. reuteri formulation was 500 μL of 2×10⁹ CFU L. reuteri, 20 mg of DM, and 28.8 mg maltose (in 1 mL saline) [“Formulation B”]. Planktonic L. reuteri was prepared similarly but without the addition of DM-maltose slurry.

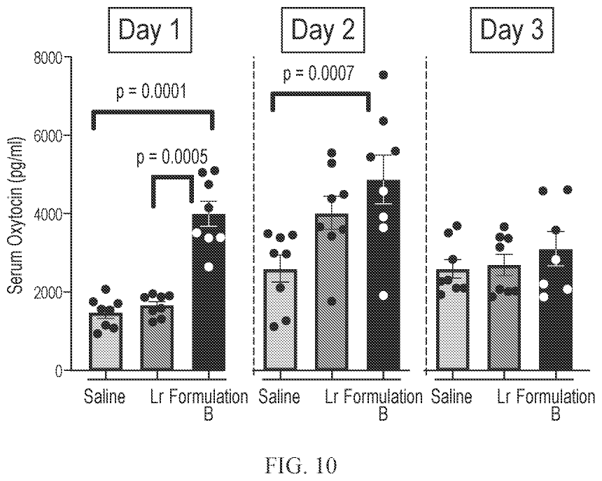

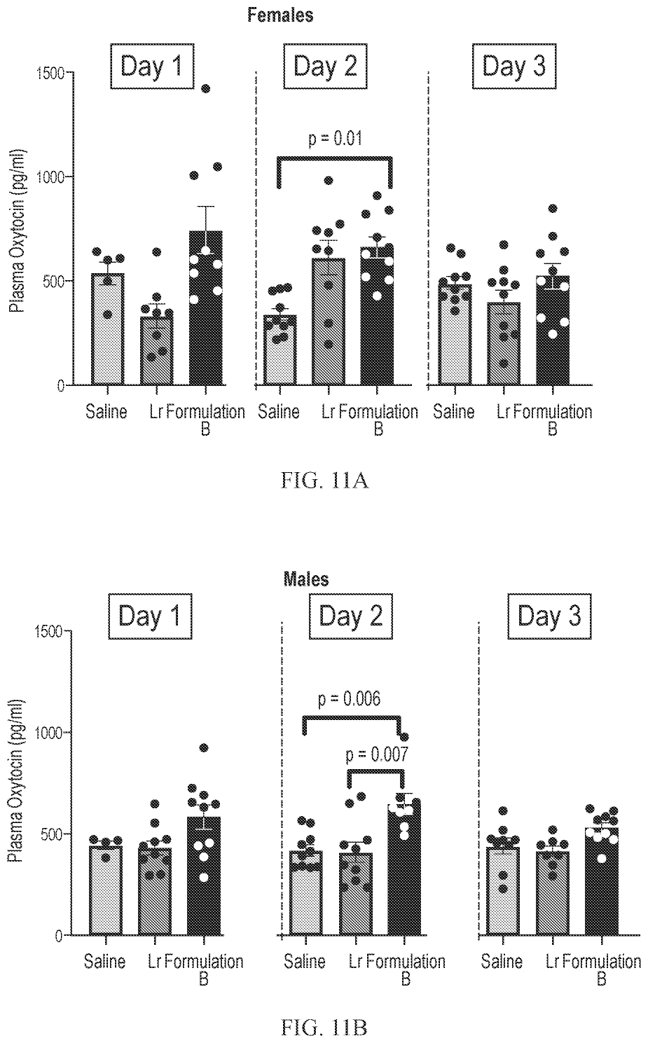

In order to investigate the effect of L. reuteri formulation on plasma oxytocin levels, adult Sprague-Dawley female rats were given a single oral dose of, planktonic L. reuteri (Lr), or saline 2 days following a single dose of prescription-grade azithromycin. (45 mg/kg, administered via oral gavage) to create a niche for L. reuteri (2-day washout period was used between the antibiotic and treatments). At day 1, 2, and 3 post-dosing, blood was collected in tubes containing aprotinin (500 Kallikrein Inactivator Units (KIU)/mL blood; used due to the short half-life of oxytocin in blood), and plasma oxytocin levels were determined by enzyme immunoassay (EIA; Assay Designs). FIG. 10 is a graph showing plasma oxytocin levels in female rats following treatment with Formulation B, planktonic L. reuteri (Lr), or saline. Oxytocin levels are shown as an average±standard error mean for the first three days post-treatment (day noted above bars). ANOVA followed by Tukey's multiple comparison was used to compare groups. As shown in FIG. 10, day 1 plasma oxytocin levels in Formulation B-treated animals were significantly higher than that of animals treated with planktonic L. reuteri or saline. Oxytocin levels between the groups leveled off by day 3. This study showed that Formulation B was effective at stimulating circulating oxytocin on day 1, which was maintained on day 2. Additionally, planktonic L. reuteri showed a trend in its ability to stimulate oxytocin levels on day 2, but levels were not significantly different than the saline-treated group.

In order to investigate the effect of L. reuteri formulation on plasma oxytocin levels in pups, pups were dosed with saline, L. reuteri, or 300 μL Formulation B (prepared as described above). Immature rat pups have low baseline circulating oxytocin levels with no sexual differences in baseline levels or in response to Formulation B. All animals were treated once via oral gavage at day 15 of age. Blood was collected on days 1, 2, and 3 post-treatment and plasma oxytocin was measured using methods described above. FIG. 11A is a graph showing plasma oxytocin levels in female pups following treatment with Formulation B, planktonic L. reuteri (Lr), or saline. FIG. 11B is a graph showing plasma oxytocin levels in male pups following treatment with Formulation B, planktonic L. reuteri (Lr), or saline. The values are shown as an average±standard error mean for the first three days post-treatment (day noted above bars). ANOVA followed by Tukey's multiple comparison test was used to compare groups. This study confirmed that Formulation B is capable of increasing circulating oxytocin levels.

### Example 7—Evaluation of L. reuteri Formulations

This example describes the impact of various preparation methods on the L. reuteri formulation including measurements of the production of lactic acid, histamine, vitamin B-12, and glycerol by L. reuteri in formulation in addition to its sugar utilization. The examples describes assessments evaluated for two L. reuteri formulations: 1) Formulation B (2×10⁹ CFU L. reuteri, 20 mg of DM, and 28.8 mg maltose (in 1 mL saline); and 2) Formulation C (that contains the same amount of L. reuteri (2×10⁹ CFU) as Formulation B, but ten times DM and maltose (200 mg DM and 288 mg maltose). The process for formulation preparation was done as described in example 6 except that the incubation of the bacteria with the DM-maltose slurry was done for various time points (1 hour, 6 hour, 24 hour, and 96 hour) prior to being assessed for various analytes in the media. Results from these studies are summarized in Table 1.

TABLE 1
Summary of evaluations of two L. reuteri formulations
(Formulation B and Formulation C)

| | | Method/ | Summary of Results | |
|---|---|---|---|---|
| Assessment | | Purpose | Formulation B | Formulation C |
| Acid pro- | pH | Standard pH | Slight decrease in pH of | Moderate decrease |
| duction | | meter | Formulation B compared | in pH over time but |
| | | | to L. reuteri alone with pH | reaching 3.5 after |
| | | | of 4.2 for | only 6 hours |
| | | | Formulation B | |
| | | | and 3.5 for L. retueri at 24 | |
| | | | hours | |
| | Lactate | Megazyme Assay kit | By 6 hours, Formulation B | Maximum lactate |
| | | | exhibited higher levels of | was produced by 6 |
| | | | lactate compared to L. | hours, Formulation |
| | | | reuteri. Lactate levels for | C exhibited higher |
| | | | Formulation B and L. | levels of lactate |
| | | | reuteri were at 24 hours | compared to L. |
| | | | were 0.35 mg/mL and | reuteri. Lactate |
| | | | 0.15 mg/mL, | levels for |
| | | | respectively. | Formulation C and |
| | | | | L. reuteri were at |
| | | | | 24 hours were 0.62 |
| | | | | mg/mL and 0.15 |
| | | | | mg/mL, |
| | | | | respectively. |
| Sugar Utilization | Maltose | Maltose and Glucose | Highest levels seen at 1 | Not determined |
| | | Assay Kit | hour with values leveling | |
| | | | off between 6 and 24 | |
| | | | hours. | |
| | Glucose | Maltose and | Slight increase was | Not |
| | | Glucose | observed in levels of | determined |
| | | Assay Kit | glucose from 1 hour to 6 | |
| | | | hours with leveling off | |
| | | | between 6 and 24 hours. | |

### Example 8—Identification of L. reuteri Formulation to Increase Plasma Oxytocin Levels

Rat pup model described in example 6 is used to identify the optimal L. reuteri formulation and dosing strategy to increase plasma oxytocin levels. Treatments with various concentrations and regimens of L. reuteri formulation are performed in order to identify the minimal efficacious dose, which defined a significant stimulation of plasma oxytocin during the initial 48 hours compared to animals treated with saline. The optimal L. reuteri formulation and dose is then assessed with activated L. reuteri formulation followed by a protocol to lyse L. reuteri to determine if similar efficacy is achieved as seen with formulation containing intact L. reuteri. The formulation that provides the minimal efficacious dose is tested in an adult nonpregnant rat to assure efficacy in a mature female.

Male and female immature rat pups at 15 days of age, are randomized to receive the following treatments. 5 male and 5 female rat pups per group are randomly assigned: 1) saline; 2) planktonic L. reuteri; or 3) L. reuteri formulation. L. reuteri formulation and planktonic L. reuteri are prepared as described in example 6 and include 2×10⁹ CFU of L. reuteri/1 mL saline. Along with 2×10⁹ CFU of L. reuteri/1 mL saline, L. reuteri formulation also include 10 mg DM with maltose at 14.4 mg, 20 mg DM with maltose at 28.8 mg, 100 mg DM with maltose at 144 mg, or 200 mg DM with maltose at 288 mg. Activation of L. reuteri formulation is done for 1 hour as described in example 6 and doses are given once via oral gavage at 300 μL. The study uses step-down approach involving first evaluation of formulation with 2×10⁹ CFU/mL L. reuteri, 200 mg DM, and 288 mg maltose. Following dosing then use formulations with the lower levels of DM and maltose, decreasing the formulation until efficacy to statistically stimulate plasma oxytocin levels is not observed. Blood is collected at 24 hours and 48 hours by tail snip and oxytocin is assayed (see Grewen, K. M. et al. (2010); Psychophysiology 47, 625-632). Groups are compared using 1-way ANOVA and Tukey's test for multiple comparisons. The minimal efficacious L. reuteri formulation is then assessed with activated L. reuteri formulation followed by a protocol to lyse L. reuteri. The lysis protocol utilizes STET buffer, which contains lysozyme to cleave the cell membrane and wall; sucrose to maintain osmotic pressure; Triton X to help cleave the cell wall, Ethylenediaminetetraacetic acid (EDTA) as a chelating agent, and Tris HCl to buffer. Intact L. reuteri formulation is compared to lysed L. reuteri formulation using male and female immature rat pups as described in the previous study (5 males and 5 females rat pups per group) and animals receiving saline or saline with STET buffer as controls. The minimal efficacious L. reuteri formulation (lysed or intact) that provides a statistically significant increase in oxytocin levels is then assessed in mature female Sprague Dawley rats to confirm that the activity seen in rat pups translates to adults. Eight female adult rats per group are treated via oral gavage (500 μl) with the L. reuteri formulation, planktonic L. reuteri, or saline and assessed for oxytocin levels at 24 hours, 48 hours, and 72 hours post-dosing as described in example 6. The minimal efficacious dose is a significant stimulation of oxytocin at 1 of 2 time points (24 hours or 48 hours). Stimulation of oxytocin may be sustained for 72 hours with the formulations containing the highest levels of DM and maltose (maximal efficacious dose). Oxytocin levels are compared to the saline-treated groups using 1-way ANOVA and Tukey's test for multiple comparisons.

### Example 9—Effect of Administering L. reuteri Formulation after Administration of Azithromycin

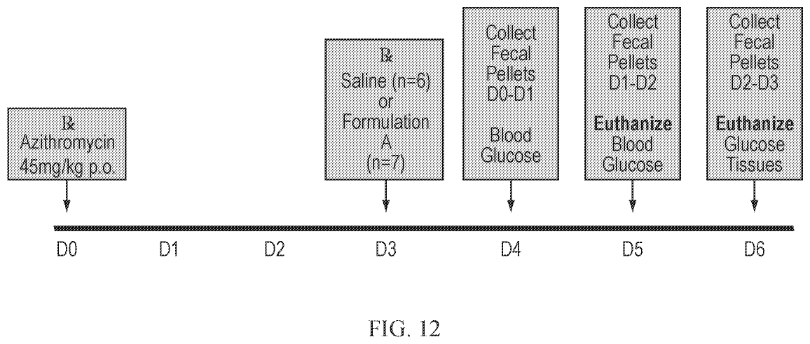

FIG. 12 illustrates the experimental study design to evaluate the effect of administering a L. reuteri formulation that includes 100 mg dextranomer microparticle (cross-linked dextran with epicholorhydine) loaded with 400 μmoles prebiotic maltose, and 1×10⁹ CFU L. reuteri (ATCC 23272) [Formulation A] on the morning of the 3rd day after administration of prescription grade Azithromycin (R Azithromycin). Briefly, Teklad 2920X chow fed-female Sprague-Dawley rats (170 g in weight) were administered [symbol] Azithromycin (45 mg/kg, oral administration) on day 0 of the experimental study. On the morning of the 3rd day after administration of [symbol] Azithromycin, 6 female rats were administered a single enteral dose of prescription grade-saline (R Saline) and 7 female rats were administered a single enteral dose of prescription grade-Formulation A (R Formulation A). Fecal pellets were collected from female rats on days 4, 5, and 6 after azithromycin but days 1, 2, and 3 after either saline or Formulation A. Blood glucose was measured on days 4, 5, and 6. Female rats were euthanized on days 5 and 6. Tissues (stomach, jejunum, ileum, cecum, proximal colon, and distal colon) were collected on day 6.

### Assessment of Stomach Content

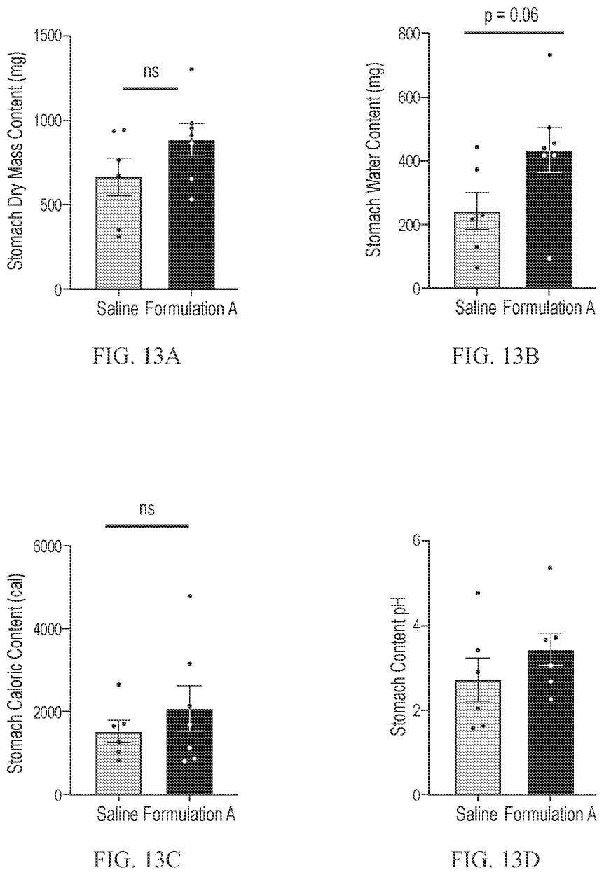

FIGS. 13A-D show the stomach content dry mass, stomach water content, stomach caloric content, and pH of stomach content on the morning of the 3rd day after administration of either [symbol] Azithromycin or [symbol] saline in saline-treated and Formulation A-treated female rats, respectively. FIG. 13B shows that administration of Formulation A to the female rats after administration of Azithromycin increased stomach water content.

### Assessment of Gut Inflammation by Analyzing Fecal Samples

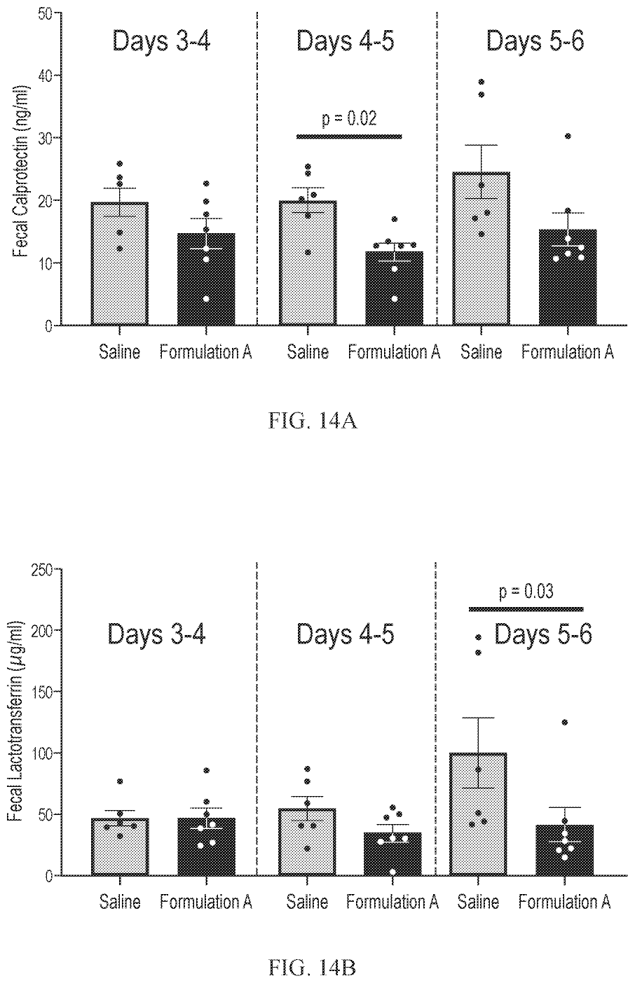

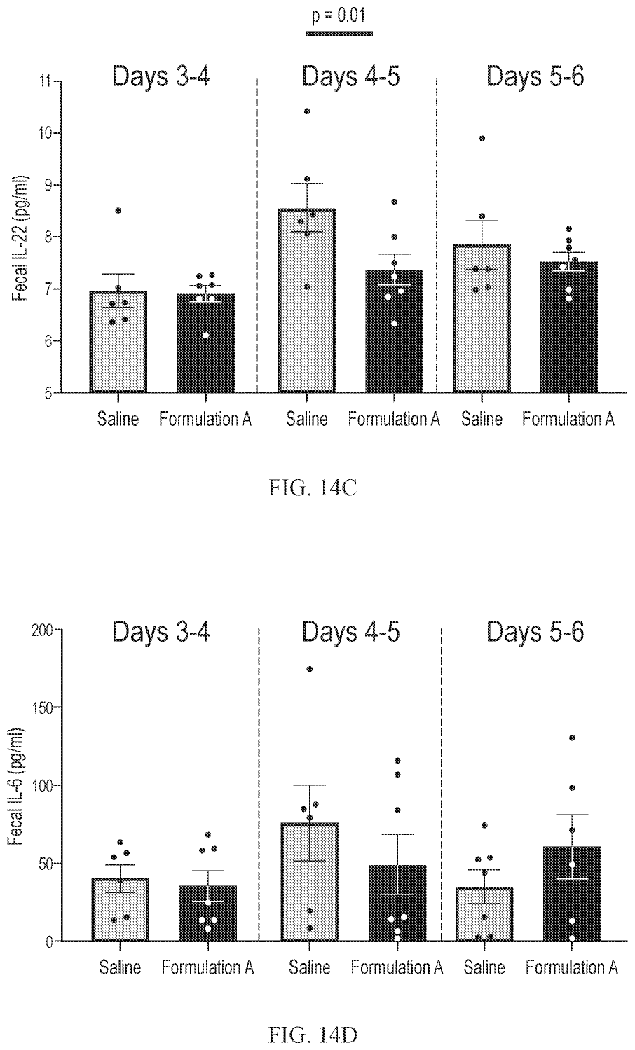

Gut inflammation can be assessed by analyzing fecal calprotectin and lactotransferrin levels. FIGS. 14A-B show fecal calprotectin and fecal lactotransferrin levels in saline-treated and Formulation A-treated female rats, respectively. FIG. 14A shows a decrease in fecal calprotectin levels in Formulation A-treated female rats as compared to saline-treated female rats on second day after administration. FIG. 14B shows a decrease in fecal lactotransferrin levels in Formulation A-treated female rats as compared to saline-treated female rats on third day after administration. FIGS. 14C-D show fecal IL-22 and fecal IL-6 levels in saline-treated and Formulation A-treated female rats, respectively. FIG. 14C shows a decrease in fecal IL-22 levels in Formulation A-treated female rats as compared to saline-treated female rats on second day after administration. FIG. 14D shows that fecal IL-6 levels were not altered by administration of Formulation A. The data presented in FIGS. 14A-D was collected between days 3 and 4, between days 4 and 5, and between days 5 and 6 after administration of [symbol] Azithromycin.

### Assessment of Pro- and Anti-Inflammatory Cytokines in the Ileum

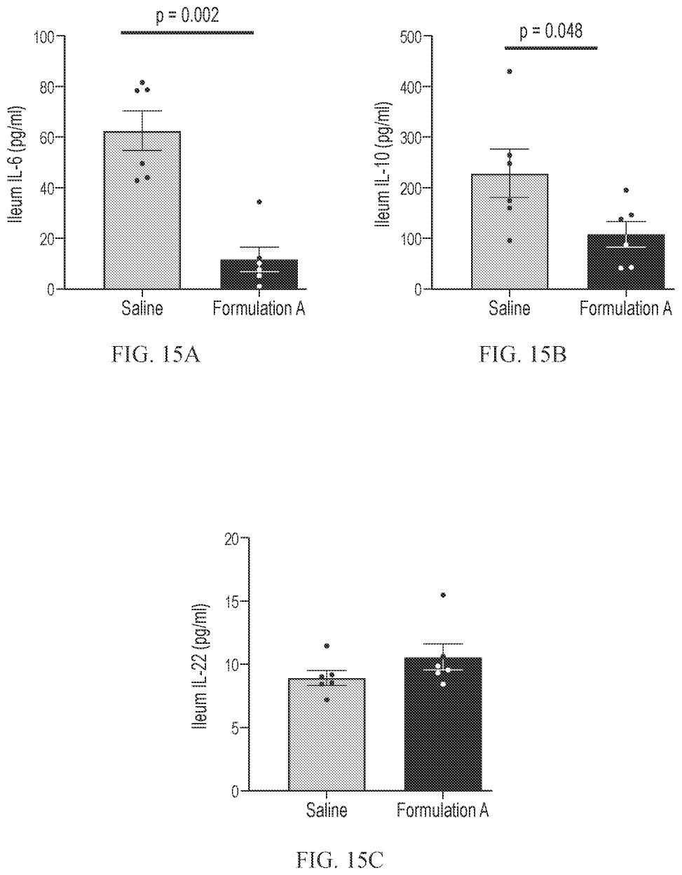

Inflammation in the ileum can be assessed using fecal markers of inflammation. IL-10 and IL-22 are anti-inflammatory cytokines. Briefly, the ileum was isolated from euthanized female rats. The contents of the ileum were harvested into a vial and were stored at −20° C. until assayed. FIGS. 15A-C show IL-6, IL-10, and IL-22 levels in ileum of saline-treated and Formulation A-treated female rats on the third day after administration of either Formulation A or saline, respectively. FIG. 15A shows that administration of Formulation A causes a decrease in levels of IL-6, a pro-inflammatory cytokine, in ileum of Formulation A-treated female rats. FIG. 15B shows that administration of Formulation A causes a decrease in levels of IL-10, an anti-inflammatory cytokine, in ileum of Formulation A-treated female rats. FIG. 15C shows that administration of Formulation A causes an increase in levels of IL-22, an anti-inflammatory cytokine, in ileum of Formulation A-treated female rats

### Assessment of Gut Repair

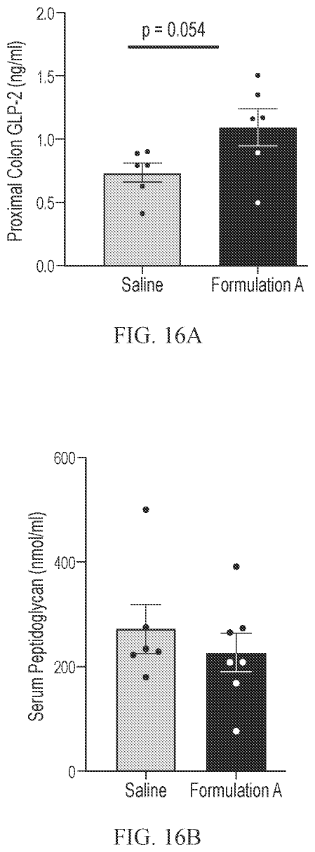

Glucagon-like peptide 2 (GLP-2) promotes tight junctions and stimulates small and large intestine growth, and transporters for glucose and fatty acid uptake. In order to assess gut repair, GLP-2 levels were measured in proximal colon. Proximal colon was obtained from euthanized female rats on third day after administration of either Formulation A or saline. FIG. 16A shows GLP-2 levels in proximal colon of saline-treated and Formulation A-treated female rats. FIG. 16A shows that administration of Formulation A causes an increase in GLP-2 levels in proximal colon.

### Assessment of Leaky Gut

Serum peptidoglycan levels were measured in Formulation A-treated and saline-treated female rats. Briefly, the adult female rats were euthanized after 3 days of administration of either [symbol] saline or Formulation A by decapitation and trunk blood was collected into tubes and allowed to clot when placed in ice. Serum was separated by centrifugation and stored at −20° C. until assayed for peptidoglycan by ELISA. FIG. 16B compares serum peptidoglycan levels in female rats on third day after administration of R saline or [symbol] Formulation A. FIG. 16B shows that administration of Formulation A did not change serum peptidoglycan levels that were elevated due to administration of Azithromycin.

### Assessment of Gut Protection

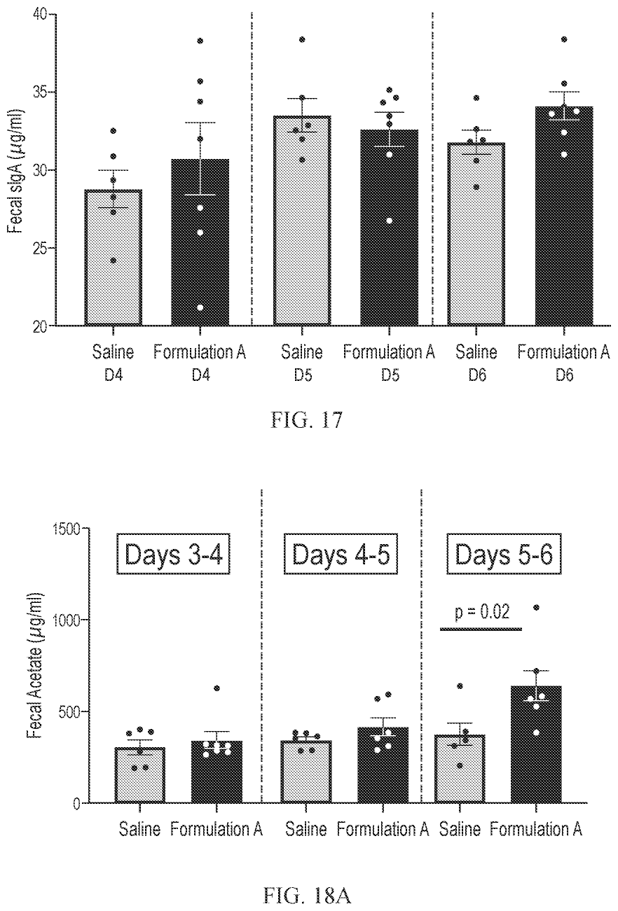

Fecal Secretory Immunoglobulin A (sIgA) provides protection against potentially pathogenic microbes, due to its ability to resist degradation by enzymes and survive in harsh environments such as gastrointestinal tract. Patients with low IgA levels have increased risk of infections in mucosal surfaces, food allergies, celiac-like enteropathies, and autoimmune disorders. Fecal sIgA levels were measured in Formulation A-treated and saline-treated female rats to assess gut protection. FIG. 17 shows fecal sIgA levels as measured on days 1, 2, and 3 after administration of either [symbol] Formulation A or [symbol] saline (or days 4, 5, and 6 after administration of [symbol] Azithromycin) in female rats to assess gut protection. FIG. 17 shows that fecal sIgA levels increase in female rats on the third day after administration of Formulation A. This increase in fecal sIgA levels in gut lumen of adult female rats after administration of Formulation A provides an evidence of gut protection.

### Assessment of SCFA Production

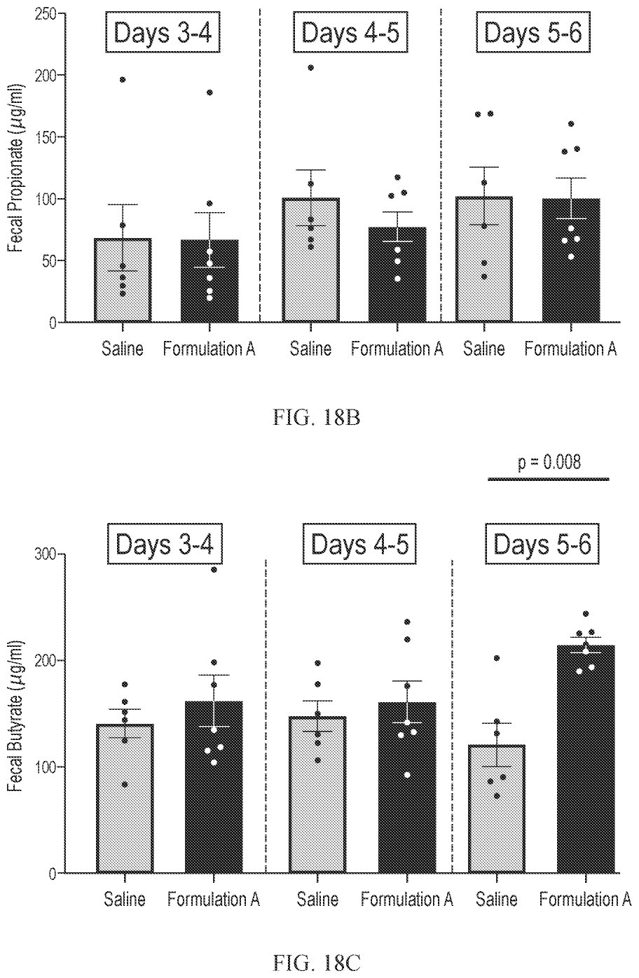

Measurement of SCFA (acetate, propionate, and butyrate) were performed by Gas chromatography-mass spectrometry (GC/MS). Briefly, fecal pellets were collected between days 3 and 4, 4 and 5, 5 and 6 from both Formulation A-treated and saline-treated female rats. The fecal pellets were stored at −20° C. until assayed. The samples were sonicated in 0.5% phosphoric acid (200 mg/ml), centrifuged to remove solid, unextracted material and filtered through 0.6 μm filter to remove any large particles. Those samples were injected (1 μl) into a GC/MS with a Stabilwax-DA column. A standard solution of 6 short chain fatty acids was used to calibrate the samples. FIGS. 18A-C compare the concentration of various SCFA (fecal acetate shown in FIG. 18A; fecal propionate shown in FIG. 18B; fecal butyrate shown in FIG. 18C) in [symbol] saline or Formulation A-treated female rats. FIGS. 18A and 18C show that acetate and butyrate levels are increased in fecal pellets excreted between days 2 and 3 after administration of Formulation A, respectively. As shown in FIG. 18B, there is no statistical difference in propionate levels between Formulation A-treated and saline-treated adult female rats.

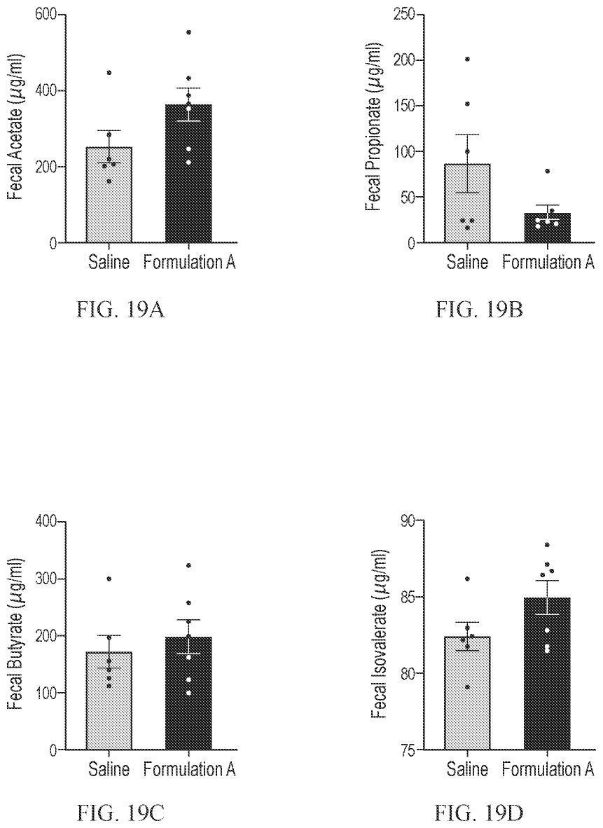

In addition to measurement of SCFA from collected fecal pellets, various SCFA levels were also measured in feces of the distal colon from euthanized female rats on third day after administration of Formulation A or saline. Briefly, fecal pellets in the distal colon were harvested into a vial and were stored at −20° C. until assayed. FIGS. 19A-D compare the concentration of various SCFA (fecal acetate shown in FIG. 19A; fecal propionate shown in FIG. 19B; fecal butyrate shown in FIG. 19C; fecal isovalerate shown in FIG. 19D) in R saline or Formulation A-treated female rats on third day after administration of either [symbol] saline or [symbol] Formulation A. As shown in FIGS. 19A and 19D, acetate and isovalerate levels were increased in female rats on third day after administration of Formulation A. FIG. 19B shows that propionate levels were decreased in female rats on third day after administration of Formulation A. As shown in FIG. 19C, there is no statistical difference in butyrate levels between Formulation A-treated and saline-treated adult female rats.

The data presented in Example 3 shows that a single dose of Formulation A significantly reduced gut inflammation after 2-3 days of administration and was effective in mitigating the side effects of Azithromycin.

### Example 10—A Study Evaluating L. reuteri Formulation

This example describes a randomized, double-blind, placebo-controlled, single-dose study to evaluate the safety and tolerability of a single dose L. reuteri formulation (L. reuteri with Sephadex® and maltose) in healthy adult volunteers. Such a study monitors the elimination of L. reuteri and Sephadex® from the gastrointestinal (GI) tract in healthy adults and includes evaluation of biomarkers of microbiota function, immune modulation, and digestion following a single oral administration of L. reuteri formulation in healthy adults.

L. reuteri Kandler (ATCC 23272) with Sephadex® and maltose is administered orally at a dose of 2×10¹⁰ colony-forming units (CFU), with 200 mg Sephadex® and 1M maltose, in a final volume of 10 mL [“Formulation B”]. L. reuteri Kandler is provided as a lyophilized powder for reconstitution in sterile saline. Following reconstitution, L. reuteri Kandler is mixed with a slurry of Sephadex® and maltose.

30 eligible subjects are randomly allocated in a 1:1 ratio to receive treatment with either Formulation B or placebo (saline). Subjects are screened within 14 days before administration of either Formulation B or placebo. All subjects are required to provide written informed consent before any study-specific procedures are performed. Subjects' eligibility for the study is determined at screening by assessment of inclusion and exclusion criteria. Subjects are asked to provide consent to allow biobanking of biological samples for future analysis. Eligible subjects are provided with a stool sampling kit to collect a pretreatment stool sample within 48 hours before receiving Formulation B or placebo (timing of collection is determined by the frequency of the subject's bowel movements).

Subjects return to the study site on Day 1 after an 8-hour overnight fast. Following confirmation of eligibility criteria, subjects receive a single oral dose of either Formulation B or placebo according to the treatment group they are allocated to, followed by 100 mL water. The fast is maintained until 1 hour after administration of either Formulation B or placebo. Subjects undergo study-specific procedures on Day 1 in accordance with the schedule of events. Vital signs (pulse rate, systolic and diastolic blood pressure, breathing rate, and oral temperature) are measured before administration of either Formulation B or placebo and at 1 hour following administration. A baseline stool sample is collected in the 48-hour window prior to the first dose for determining the presence of L. reuteri and Sephadex® microspheres and exploratory biomarker analysis. Subjects are discharged after all Day 1 evaluations have been completed. Subjects are instructed to collect 4 stool samples at home according to the following schedule: Subjects are provided with sampling kits to collect stool samples at home. Kits include labels for sample containers, which are completed with the time and date that the sample was obtained. Subjects are instructed to return all stool samples using pre-labelled shipping containers; alternative strategies for returning stool samples can also be discussed by the investigator and subjects.

Follow-up visits are conducted on Days 8 and 15. Subjects attend an End-of-study Visit on Day 30.

Eligible subjects receive a single dose of either Formulation B or placebo according to the treatment group to which they are allocated. Either Formulation B or placebo is administered at the study site on Day 1 under fasting conditions. Following administration, subjects are provided 100 mL water to drink. Both Formulation B and placebo are presented to the subject in an opaque plastic syringe so the contents are not visible, to maintain the study blind. Subjects are required to fast for at least 8 hours before administration of either Formulation B or placebo. Subjects are required to refrain from using other probiotics and any antibiotics for the duration of their study involvement. Safety endpoints include incidence and severity of treatment-emergent AEs (TEAEs), serious AEs (SAES), AESIs, and AEs leading to discontinuation from the study; Laboratory results (hematology [complete blood cell count with differential and erythrocyte sedimentation rate], biochemistry, and urinalysis), vital signs results, and physical examination findings; Incidence of presence of L. reuteri Kandler, Sephadex® microspheres, and leukocytes in the stool; Bristol Stool chart scores; Incidence of symptomatic bacteremia with positive L. reuteri identification. Other endpoints for the study include: Biomarkers for microbiota function: Short-chain fatty acids (acetate, butyrate, propionate), lactate, and pH; Biomarkers for immune modulation: Proinflammatory and anti-inflammatory cytokines (interferon-γ, interleukin [IL]-2, IL-4, IL-5, IL-6, IL-10, IL-22, chemokine ligand 1, and tumor necrosis factor-α), as well as calprotectin, lactoferrin, and secretory immunoglobulin A; Biomarkers for digestion: Fecal content of glucose, triglycerides, total bile acids, protein, lactose.

Demographic and baseline characteristics (including age, sex, race, ethnicity, weight, height, and body mass index) are summarized using descriptive statistics and qualitative assessments of levels of L. reuteri, Sephadex® microspheres, and leukocytes in stool samples are conducted, with results summarized using descriptive statistics.

The overall study duration includes 1 month of active enrollment and 1 month of follow-up. The sequence and maximum duration of the study periods is as follows: Screening: 14 days; Treatment: 1 day; Follow-up: 30 days. The maximum study duration for each subject is approximately up to 6 weeks. The maximum treatment duration for each subject is 1 day.

### Example 11—Effect of Administering L. reuteri Formulation on Autistic Disorders

The example details the study design evaluating the effect of administering L. reuteri formulation in the treatment of autistic disorders. In comparison to premature infants at risk of developing NEC born with immature and underdeveloped gastrointestinal (GI) systems, the GI system of the target population is much more well developed (see Yatsunenko, T. et al. (2012); Nature.; 486, 222-227). However, GI symptoms, including constipation, abdominal pain, flatulence, and diarrhea, are often associated with autistic disorder in a prevalence range from 23% to 70% (see Chaidez, V. et al. (2014); Journal of autism and developmental disorders.; 44(5), 1117-1127; Holingue, Calliope et al. (2018); Autism research: official journal of the International Society for Autism Research.; 11(1), 24-36). Neither GI pathology after histological examination nor changes in fecal consistency and fecal output in preclinical studies using L. reuteri formulation have been observed. Moreover, a decrease in fecal calprotectin levels, a clinical biomarker of GI inflammation, has been observed, suggesting that L. reuteri formulation may reduce clinical or subclinical gut inflammation and may improve GI symptoms commonly associated with autistic disorder.

Following initial screening, the subjects are randomized to one of 2 groups of 8 subjects each. Group A receives L. reuteri formulation for 28 days while Group B receives a placebo (saline). Following initial dosing, there is a 14 day washout period during which the subjects receive neither L. reuteri formulation nor placebo. Following the washout period, Group A receives a placebo (saline) for 28 days, while Group B will receives L. reuteri formulation. The primary outcome is to assess the safety and tolerability of L. reuteri formulation administered daily over a period of 28 days as assessed by the frequency and severity of adverse events and laboratory abnormalities. Secondary outcomes include assessments of the effects of L. reuteri formulation on circulating oxytocin levels, systemic and fecal levels of host and microbially-derived metabolites, blood and fecal biomarkers of gut inflammation, and validated instruments for behavior in the context of autistic disorder. Total study duration is approximately 70 days.

A subject is eligible for study participation if the subject has a diagnostic confirmation of Autistic Disorder as confirmed by gold standard clinical interview using DSM-5 criteria and administration of the Autism Diagnostic Observation Schedule-2. L. reuteri Kandler is administered orally at a dose of 2×1010 colony forming units, with 185 mg Sephadex® and 74 mM maltose, in a final volume of 10 mL. Subjects receive a single dose a day for 28 days. L. reuteri Kandler is provided as a lyophilized powder for reconstitution in saline (provided). Following reconstitution, the L. reuteri Kandler is mixed with a slurry of Sephadex® and maltose. The reference product/placebo is saline. Subjects receive a single oral dose of 10 mL matched saline for 28 days.

Eligible subjects receive one dose of L. reuteri Kandler or placebo daily for 28 days according to the treatment group to which they are allocated. 28 doses are provided to the subject, prepared at home and combined with a preferred drink or soft food (e.g. applesauce, yogurt, pudding). The first dose of IP is administered at the clinic. Subjects are required to refrain from using probiotics for the duration of their study involvement.

The clinician and caregiver report outcome measure analysis consists of two paired t-tests between L. reuteri Kandler versus placebo. The measured value for each subject is the change in continuous measures including the CGI-S, CGI-I, ABC, CFQL-2, WJ3 Spatial Relations and Auditory Attention subtests, KiTap, RBANS, eye tracking, and psychophysical measures. For each subject the difference between the changes in a continuous outcome measure score from the change for placebo is derived. A one-sample (paired) t-test is conducted for these intra-subject differences. A result is considered statistically significant if the two-sided p-value is less than 0.05 with correction for multiplicity using the False Discovery Rate (FDR) approach. In addition, a linear mixed model for the two-treatment crossover design is analyzed. The response for this model is the continuous outcome measure of interest at the end of the period. The fixed effect covariates are treatment, period, gender, and carry-over.

The sequence and maximum duration of the study periods is as follows: Screening: 14 days; Treatment period 1: 28 days; Washout: 14 days; Treatment period 2: 28 days; Follow-up: 30 days; study duration for each subject is approximately up to 17 weeks. Eye tracking measurements are taken at either the screening or baseline visit for participants with ASD. ADOS-2=Autism Diagnostic Observation Schedule Module 3 or 4; WASI-II=Wechsler Abbreviated Scale of Intelligence Scale-Second Edition,_SCQ=social communication questionnaire; CGI-S=clinical global impressions severity scale, CGI-I=clinical global impressions improvement scale; Vineland-3=Vinland Adaptive Behaviors Scales 3^{rd} edition; PK=pharmacokinetics; EEG=electroencephalogram protocol; WJ3=Woodcock Johnson Spatial Relations and Auditory Attention subtests; RBANS=repeatable battery of neuropsychological status; KiTap=computerized test of attentional performance in children; ABC=aberrant behavior checklist; CFQL-2=Child and Family Quality of Life 2nd Ed.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

### INCORPORATION BY REFERENCE

The entire contents of all patents, published patent applications, websites, and other references cited herein are hereby expressly incorporated herein in their entireties by reference.

1. A method of treating or ameliorating an autism spectrum disorder in a subject in need thereof, the method comprising administering to the subject a therapeutically effective amount of a composition comprising Lactobacillus reuteri, a biocompatible microsphere, and optionally a prebiotic.

2. The method of claim 1, wherein the autism spectrum disorder is selected from the group consisting of autistic disorder, Asperger syndrome, Heller's syndrome, Rett syndrome, and pervasive developmental disorder, and not otherwise specified (PDD-NOS).

3. The method of any one of claims 1-2, wherein the composition comprises a dextranomer microparticle; L. reuteri, ATCC 23272; and a prebiotic.

4. The method of any one of claims 1-3, wherein upon administration of at least one daily dose of the composition to the subject, the subject has an upregulation in oxytocin levels.

5. The method of any one of claims 1-4, wherein the subject is a pediatric patient.

6. The method of any one of claims 1-5, wherein the subject also has a gastrointestinal disorder.

7. A method of treating a patient for a gastrointestinal disorder, comprising administering a composition comprising dextranomer microparticles; L. reuteri, ATCC 23272; and a prebiotic.

8. The method of claim 7, wherein the patient is on the autism spectrum or is autistic.

9. The method of claim 7 or 8, wherein the gastrointestinal disorder is associated with antibiotic administration.

10. A method of treating an autistic patient suffering gastrointestinal disorders, comprising administering a composition comprising: dextranomer microparticles; L. reuteri, ATCC 23272; and a prebiotic.

11. The method of any one of claims 7-10, wherein the gastrointestinal disorder is one or more of constipation, abdominal pain, flatulence, and diarrhea.

12. A method of treating a developmental disorder which is associated with preterm birth in a preterm infant, the method comprising administering to the preterm infant a therapeutically effective amount of a composition comprising Lactobacillus reuteri and a pharmaceutically acceptable carrier.

13. A method of inducing oxytocin in a preterm infant in need thereof, the method comprising administering to the preterm infant a therapeutically effective amount of a composition comprising Lactobacillus reuteri and a pharmaceutically acceptable carrier.

14. The method of claim 12 or 13, wherein the composition further comprises a biocompatible microsphere, and/or a prebiotic.

15. The method of any one of claims 12-14 wherein the composition comprises a dextranomer microparticle, L. reuteri, ATCC 23272, and a prebiotic.

16. The method of any one of claims 12-14, wherein upon administration of at least one daily dose of the composition to the preterm infant, the preterm infant has an upregulation in oxytocin levels.

17. A method of treating gastrointestinal inflammation in a patient due to administration of an antibiotic regimen, comprising administering to the patient a therapeutically effective amount of a composition comprising Lactobacillus reuteri, a biocompatible microsphere, and/or a prebiotic.

18. A method of treating a patient having or expected to have gastrointestinal inflammation associated with administration of an oral antibiotic regimen, comprising administering to the patient a therapeutically effective amount of a composition comprising Lactobacillus reuteri, a biocompatible microsphere, and/or a prebiotic.

19. The method of claim 17 or 18, wherein the composition is administered before administration of the antibiotic regimen, during the administration of the antibiotic regimen, and/or after administration of the antibiotic regimen.

20. A method of substantially preventing or decreasing an oral antibiotic-associated adverse effect in a subject in need thereof, comprising administering a therapeutically effective amount of a composition comprising Lactobacillus reuteri, a biocompatible microsphere, wherein the subject is undergoing treatment with the oral antibiotic.

21. A method of treating depression or an anxiety disorder in a subject in need thereof, the method comprising administering to the subject a therapeutically effective amount of a composition comprising Lactobacillus reuteri, a biocompatible microsphere, and/or a prebiotic.

22. The method of claim 21, wherein depression is selected from the group consisting of clinical depression, postnatal or postpartum depression, obsessive-compulsive disorder, post-traumatic stress disorder, bipolar disorder, atypical depression, melancholic depression, Psychotic Major Depression (PMD), catatonic depression, Seasonal Affective Disorder (SAD), dysthymia, double depression, Depressive Personality Disorder (DPD), Recurrent Brief Depression (RBD), minor depressive disorder, bipolar disorder or manic depressive disorder, treatment-resistant depression, refractory depression, suicidality, suicidal ideation, and suicidal behavior.

23. The method of claim 21, wherein the patient has postnatal or postpartum depression.

24. The method of any one of claims 1-23, wherein the composition is administered once.

25. The method of any one of claims 1-23, wherein the composition is administered daily.

26. The method of claims 1-25, wherein the prebiotic comprises a water-soluble carbohydrate, wherein the water-soluble carbohydrate comprises one or more of inulin, oligofructose, fructo-oligosaccharide, galacto-oligosaccharide, glucose, starch, maltose, maltodextrins, polydextrose, amylose, sucrose, fructose, lactose, isomaltulose, polyols, glycerol, carbonate, thiamine, choline, histidine, trehalose, nitrogen, sodium nitrate, ammonium nitrate, phosphorus, phosphate salts, hydroxyapatite, potassium, potash, sulfur, homopolysaccharide, heteropolysaccharide, cellulose, chitin, vitamins, or a combination thereof.

27. The method of any one of claims 1-26, wherein the prebiotic is maltose.
